(19) United States
(12) Patent Application Publication
(10) Pub. No.: US 2023/0270739 A1
(43) Pub. Date: Aug. 31, 2023

(54) Methods and Compositions for Treating Pain While Minimizing the Risk of Opioid Addiction

(71) Applicants: The Regents of the University of California, Oakland, CA (US); The United States Government as Represented by the Department of Veterans Affairs, 810 Vermont Ave., NW, DC (US)

(72) Inventors: Jerome Siegel, Porter Ranch, CA (US); Ronald McGregor, Sherman Oaks, CA (US); Ming-Fung Frank Wu, Porter Ranch, CA (US)

(21) Appl. No.: 17/925,775

(22) PCT Filed: May 21, 2021
(86) PCT No.: PCT/US2021/033716

§ 371 Date: Nov. 16, 2022

Related U.S. Application Data
(60) Provisional application No. 63/029,783, filed on May 26, 2020.

(51) Int. Cl. A61K 31/485; A61K 31/5513; A61K 9/00; A61P 25/04

(52) U.S. Cl. CPC A61K 31/485; A61K 31/5513; A61K 9/0019; A61P 25/04

(57) ABSTRACT

Disclosed herein are compositions and methods for treating subjects for pain with opioids while reducing or inhibiting their risk of opioid addiction.

### CROSS-REFERENCE TO RELATED APPLICATIONS

This application claims the benefit of U.S. Patent Application No. 63/029,783, filed May 26, 2020, which is herein incorporated by reference in its entirety.

### ACKNOWLEDGEMENT OF GOVERNMENT SUPPORT

This invention was made with Government support under Grant Number DA034748, awarded by the National Institutes of Health. The Government has certain rights in the invention.

This work was supported by the U.S. Department of Veterans Affairs, and the Federal Government has certain rights in the invention.

### BACKGROUND OF THE INVENTION

### 1. Field of the Invention

The field generally relates to methods and compositions for treating pain with opioids while minimizing the risk of opioid use disorders.

### 2. Description of the Related Art

While analgesics such as nonsteroidal anti-inflammatory drugs are highly effective in relieving relatively mild pain, they do not provide nearly the relief that opioids do for severe pain. In 2018 this group discovered that the brains of human heroin addicts had an average 54% increase in the number of “detectable” hypocretin (“Hcrt” or “orexin”) neurons and a 22% shrinkage in the cross-sectional area of these neurons. These changes outlast drug intake for as long as 3 years. Similar changes are induced by long-term administration of addictive doses of morphine to mice. These changes are not a result of neurogenesis. It was subsequently found that cocaine addicted rats have the same abnormality in the number and size of Hcrt neurons, indicating that the size and number changes in Hcrt neurons may be a general correlate of addiction. No other change of brain morphology of this magnitude has been detected in addiction.

Addiction and withdrawal: The annual death rate from opiate/opioid overdoses in the US has grown exponentially, now exceeding 42,000, greater than the annual rates for automobile or gun deaths. This compares to the 8,000 level recorded before 1990. Physicians, who were previously told that it is medical malpractice to undertreat pain, are now told that they must avoid opioid prescriptions whenever possible because of the risk of dependence/addiction. Over 2 million Americans have an opioid use disorder (OUD). About 10% of addicts saved from an overdose by naloxone are dead within a year from a subsequent opioid overdose. Intravenous opioid users are also much more likely to die of HIV, hepatitis, staph, botulism, tetanus, endocarditis and other infectious disorders not included in the 42,000 toll. A critical issue in stemming deaths from OUD is the inability of many addicts to successfully withdraw from opioid use. A large proportion of OUD cases begin with the prescribed use of opioids for relief of severe pain and progresses to illegal pill acquisition or to heroin use. Although non-opioid analgesics can be used for relatively minor pain, severe burns, cancer, joint inflammation, sickle cell anemia, and other painful afflictions often cannot be effectively treated with non-opioid analgesics. These disorders cause immense suffering and can drive patients to suicide if not adequately treated.

The difficulty of withdrawal for those with OUD is not caused solely by the seeking of a pleasurable “high.” It is also due to seeking relief from the symptoms induced by withdrawal. Acute symptoms typically peak 24-48 hours after withdrawing from short-acting opioids (e.g., heroin or oxycodone). These acute symptoms may be followed by anhedonia, fatigue, anorexia, depression and insomnia, effects that persist for weeks to months or years in humans. These short and long-term effects drive most subjects who have attempted withdrawal to relapse within one year, even after medically supervised methadone, buprenorphine, or other pharmacological treatments.

### SUMMARY OF THE INVENTION

In some embodiments, the present invention is directed to a composition comprising or consisting essentially of one or more opioids, and one or more hypocretin/orexin receptor antagonists. In some embodiments, the one or more opioids is provided in a therapeutically effective amount for treating, inhibiting, or reducing pain in a subject and/or the one or more hypocretin/orexin receptor antagonists is provided in a therapeutically effective amount for inhibiting or reducing the likelihood that the subject will develop an addiction to the one or more opioids. In some embodiments, the one or more opioids is selected from opioid peptides such as endorphins, enkephalins, dynorphins, and endomorphins; opium alkaloids such as codeine, morphine, thebaine, oripavine, and papaveretum; esters of morphine such as diacetylmorphine (morphine diacetate; heroin), nicomorphine (morphine dinicotinate), dipropanoylmorphine (morphine dipropionate), diacetyldihydromorphine, acetylpropionylmorphine, desomorphine, methyldesorphine, and dibenzoylmorphine; ethers of morphine such as dihydrocodeine, ethylmorphine, and heterocodeine; synthetic alkaloids such as buprenorphine, etorphine, hydrocodone, hydromorphone, oxycodone, and oxymorphone; and synthetic opioids such as anilidopiperidines (e.g., fentanyl, alphamethylfentanyl, alfentanil, sufentanil, remifentanil, carfentanyl, ohmefentanyl), phenylpiperidines (e.g., pethidine (meperidine), ketobemidone, MPPP, allylprodine, prodine, PEPAP, promedol), diphenylpropylamine derivatives (e.g., propoxyphene, dextropropoxyphene, dextromoramide, bezitramide, piritramide, methadone, dipipanone, levomethadyl acetate, difenoxin, diphenoxylate, loperamide), benzomorphan derivatives (e.g., dezocine, pentazocine, phenazocine), oripavine derivatives (e.g., buprenorphine, dihydroetorphine, etorphine), morphinan derivatives (e.g., butorphanol, nalbuphine, levorphanol, levomethorphan, racemethorphan), lefetamine, menthol, meptazinol, mitragynine, tilidine, tramadol, tapentadol, eluxadoline, AP-237, and 7-hydroxymitragynine. In some embodiments, the one or more opioids is selected from morphine, esters of morphine, ethers of morphine, synthetic opioids, and synthetic alkaloids. In some embodiments, the one or more opioids is morphine, oxymorphone, hydromorphone, fentanyl, hydrocodone, oxycodone, oxymorphone, or hydromorphone. In some embodiments, the one or more hypocretin/orexin receptor antagonists is selected from suvorexant, almorexant, EMPA, filorexant, JNJ-10397049, lemborexant, MIN-202, MK-1064, MK-8133, nemorexant, RTIOX-276, SB-334867, SB-408124, SB-649868 (CAS No. 380899-24-1), TCS-0X2-29, (3,4-dimethoxyphenoxy) alkylamino acetamides, and Compound 1 m. In some embodiments, the one or more hypocretin/orexin receptor antagonists is suvorexant. In some embodiments, the one or more opioids is selected from morphine, esters of morphine, and ethers of morphine and the one or more hypocretin/orexin receptor antagonists is suvorexant. In some embodiments, the one or more opioids is morphine and the one or more hypocretin/orexin receptor antagonists is suvorexant. In some embodiments, the composition comprises about 1-10 mg of the one or more opioids. In some embodiments, the composition comprises about 1-10 mg of the one or more hypocretin/orexin receptor antagonists. In some embodiments, the composition comprises about 1-10 mg of the one or more opioids and about 1-10 mg of the one or more hypocretin/orexin receptor antagonists. In some embodiments, the composition is an oral formulation. In some embodiments, the composition is an intravenous formulation. In some embodiments, the compositions further comprises a pharmaceutically acceptable vehicle.

In some embodiments, the present invention is directed to a method for treating, inhibiting, or reducing pain in a subject, which comprises administering to the subject one or more opioids in combination with one or more hypocretin/orexin receptor antagonists. In some embodiments, the one or more opioids is administered in a therapeutically effective amount for treating, inhibiting, or reducing pain in a subject and/or the one or more hypocretin/orexin receptor antagonists is administered in a therapeutically effective amount for inhibiting or reducing the likelihood that the subject will develop an addiction to the one or more opioids. In some embodiments, the one or more opioids is selected from opioid peptides such as endorphins, enkephalins, dynorphins, and endomorphins; opium alkaloids such as codeine, morphine, thebaine, oripavine, and papaveretum; esters of morphine such as diacetylmorphine (morphine diacetate; heroin), nicomorphine (morphine dinicotinate), dipropanoylmorphine (morphine dipropionate), diacetyldihydromorphine, acetylpropionylmorphine, desomorphine, methyldesorphine, and dibenzoylmorphine; ethers of morphine such as dihydrocodeine, ethylmorphine, and heterocodeine; synthetic alkaloids such as buprenorphine, etorphine, hydrocodone, hydromorphone, oxycodone, and oxymorphone; and synthetic opioids such as anilidopiperidines (e.g., fentanyl, alphamethylfentanyl, alfentanil, sufentanil, remifentanil, carfentanyl, ohmefentanyl), phenylpiperidines (e.g., pethidine (meperidine), ketobemidone, MPPP, allylprodine, prodine, PEPAP, promedol), diphenylpropylamine derivatives (e.g., propoxyphene, dextropropoxyphene, dextromoramide, bezitramide, piritramide, methadone, dipipanone, levomethadyl acetate, difenoxin, diphenoxylate, loperamide), benzomorphan derivatives (e.g., dezocine, pentazocine, phenazocine), oripavine derivatives (e.g., buprenorphine, dihydroetorphine, etorphine), morphinan derivatives (e.g., butorphanol, nalbuphine, levorphanol, levomethorphan, racemethorphan), lefetamine, menthol, meptazinol, mitragynine, tilidine, tramadol, tapentadol, eluxadoline, AP-237, and 7-hydroxymitragynine. In some embodiments, the one or more opioids is selected from morphine, esters of morphine, ethers of morphine, synthetic opioids, and synthetic alkaloids. In some embodiments, the one or more opioids is morphine, oxymorphone, hydromorphone, fentanyl, hydrocodone, oxycodone, oxymorphone, or hydromorphone. In some embodiments, the one or more hypocretin/orexin receptor antagonists is selected from suvorexant, almorexant, EMPA, filorexant, JNJ-10397049, lemborexant, MIN-202, MK-1064, MK-8133, nemorexant, RTIOX-276, SB-334867, SB-408124, SB-649868 (CAS No. 380899-24-1), TCS-0X2-29, (3,4-dimethoxyphenoxy) alkylamino acetamides, and Compound 1 m. In some embodiments, the one or more hypocretin/orexin receptor antagonists is suvorexant. In some embodiments, the one or more opioids is selected from morphine, esters of morphine, and ethers of morphine and the one or more hypocretin/orexin receptor antagonists is suvorexant. In some embodiments, the one or more opioids is morphine and the one or more hypocretin/orexin receptor antagonists is suvorexant. In some embodiments, about 1-10 mg of the one or more opioids is administered. In some embodiments, about 1-10 mg of the one or more hypocretin/orexin receptor antagonists is administered. In some embodiments, about 1-10 mg of the one or more opioids and about 1-10 mg of the one or more hypocretin/orexin receptor antagonists is administered. In some embodiments, the one or more opioids and/or the one or more hypocretin/orexin receptor antagonists is administered orally. In some embodiments, the one or more opioids and/or the one or more hypocretin/orexin receptor antagonists is administered intravenously. In some embodiments, the one or more hypocretin/orexin receptor antagonists is administered before, during, or after the administration of the one or more opioids. In some embodiments, the one or more hypocretin/orexin receptor antagonists is administered within 6 hours, within 5 hours, within 4 hours, within 3 hours, within 2 hours, within 1 hour, within 30 minutes, or within 15 minutes of the administration of the one or more opioids. In some embodiments, the one or more hypocretin/orexin receptor antagonists is administered 6 hours, 5 hours, 4 hours, 3 hours, 2 hours, 1 hour, 30 minutes, or 15 minutes before the administration of the one or more opioids. In some embodiments, the one or more hypocretin/orexin receptor antagonists is administered with the one or more opioids. In some embodiments, about 0.05-0.15 mg of the one or more opioids per kg weight of the subject is administered. In some embodiments, about 0.05-0.15 mg of the one or more hypocretin/orexin receptor antagonists per kg weight of the subject is administered. In some embodiments, about 0.05-0.15 mg of the one or more opioids per kg weight of the subject and about 0.05-0.15 mg of the one or more hypocretin/orexin receptor antagonists per kg weight of the subject is administered. In some embodiments, the one or more opioids and the one or more hypocretin/orexin receptor antagonists is administered in the form of a composition as described herein, e.g., as described above.

In some embodiments, the present invention is directed to a method of inhibiting or reducing an increase in the amount of hypocretin and/or an increase in the amount of hypocretin neurons, which increases are caused by administration of one or more opioids, in a subject, which comprises administering to the subject one or more hypocretin/orexin receptor antagonists before, during, or after the administration of the one or more opioids. In some embodiments, the one or more hypocretin/orexin receptor antagonists is administered within 6 hours, within 5 hours, within 4 hours, within 3 hours, within 2 hours, within 1 hour, within 30 minutes, or within 15 minutes of the administration of the one or more opioids. In some embodiments, the one or more hypocretin/orexin receptor antagonists is administered 6 hours, 5 hours, 4 hours, 3 hours, 2 hours, 1 hour, 30 minutes, or 15 minutes before the administration of the one or more opioids. In some embodiments, the one or more hypocretin/orexin receptor antagonists is administered with the one or more opioids. In some embodiments, the one or more opioids is selected from opioid peptides such as endorphins, enkephalins, dynorphins, and endomorphins; opium alkaloids such as codeine, morphine, thebaine, oripavine, and papaveretum; esters of morphine such as diacetylmorphine (morphine diacetate; heroin), nicomorphine (morphine dinicotinate), dipropanoylmorphine (morphine dipropionate), diacetyldihydromorphine, acetylpropionylmorphine, desomorphine, methyldesorphine, and dibenzoylmorphine; ethers of morphine such as dihydrocodeine, ethylmorphine, and heterocodeine; synthetic alkaloids such as buprenorphine, etorphine, hydrocodone, hydromorphone, oxycodone, and oxymorphone; and synthetic opioids such as anilidopiperidines (e.g., fentanyl, alphamethylfentanyl, alfentanil, sufentanil, remifentanil, carfentanyl, ohmefentanyl), phenylpiperidines (e.g., pethidine (meperidine), ketobemidone, MPPP, allylprodine, prodine, PEPAP, promedol), diphenylpropylamine derivatives (e.g., propoxyphene, dextropropoxyphene, dextromoramide, bezitramide, piritramide, methadone, dipipanone, levomethadyl acetate, difenoxin, diphenoxylate, loperamide), benzomorphan derivatives (e.g., dezocine, pentazocine, phenazocine), oripavine derivatives (e.g., buprenorphine, dihydroetorphine, etorphine), morphinan derivatives (e.g., butorphanol, nalbuphine, levorphanol, levomethorphan, racemethorphan), lefetamine, menthol, meptazinol, mitragynine, tilidine, tramadol, tapentadol, eluxadoline, AP-237, and 7-hydroxymitragynine. In some embodiments, the one or more opioids is selected from morphine, esters of morphine, ethers of morphine, synthetic opioids, and synthetic alkaloids. In some embodiments, the one or more opioids is morphine, oxymorphone, hydromorphone, fentanyl, hydrocodone, oxycodone, oxymorphone, or hydromorphone. In some embodiments, the one or more hypocretin/orexin receptor antagonists is selected from suvorexant, almorexant, EMPA, filorexant, JNJ-10397049, lemborexant, MIN-202, MK-1064, MK-8133, nemorexant, RTIOX-276, SB-334867, SB-408124, SB-649868 (CAS No. 380899-24-1), TCS-OX2-29, (3,4-dimethoxyphenoxy) alkylamino acetamides, and Compound 1 m. In some embodiments, the one or more hypocretin/orexin receptor antagonists is suvorexant. In some embodiments, the one or more opioids is selected from morphine, esters of morphine, and ethers of morphine and the one or more hypocretin/orexin receptor antagonists is suvorexant. In some embodiments, the one or more opioids is morphine and the one or more hypocretin/orexin receptor antagonists is suvorexant. In some embodiments, about 1-10 mg of the one or more opioids is administered. In some embodiments, about 1-10 mg of the one or more hypocretin/orexin receptor antagonists is administered. In some embodiments, about 1-10 mg of the one or more opioids and about 1-10 mg of the one or more hypocretin/orexin receptor antagonists is administered. In some embodiments, the one or more opioids and/or the one or more hypocretin/orexin receptor antagonists is administered orally. In some embodiments, the one or more opioids and/or the one or more hypocretin/orexin receptor antagonists is administered intravenously. In some embodiments, about 0.05-0.15 mg of the one or more opioids per kg weight of the subject is administered. In some embodiments, about 0.05-0.15 mg of the one or more hypocretin/orexin receptor antagonists per kg weight of the subject is administered. In some embodiments, about 0.05-0.15 mg of the one or more opioids per kg weight of the subject and about 0.05-0.15 mg of the one or more hypocretin/orexin receptor antagonists per kg weight of the subject are administered. In some embodiments, the one or more opioids and the one or more hypocretin/orexin receptor antagonists is administered in the form of a composition as described herein, e.g., as described above.

In some embodiments, the present invention is directed to the use of one or more opioids in combination with one or more hypocretin/orexin receptor antagonists. In some embodiments, the present invention is directed to use of the combination of one or more opioids and one or more hypocretin/orexin receptor antagonists in the manufacture of a medicament for the (a) treatment, inhibition, or reduction of pain in a subject, or (b) inhibition or reduction of an increase in the amount of hypocretin and/or an increase in the amount of hypocretin neurons in a subject, which increases are caused by administration of one or more opioids. In some embodiments, the present invention is directed to use of the combination of one or more opioids and one or more hypocretin/orexin receptor antagonists to (a) treat, inhibit, or reduce pain in a subject and/or (b) inhibit or reduce an increase in the amount of hypocretin and/or an increase in the amount of hypocretin neurons in a subject, which increases are caused by administration of one or more opioids. In some embodiments, the present invention is directed to the combination of one or more opioids and one or more hypocretin/orexin receptor antagonists for use the (a) treatment, inhibition, or reduction of pain in a subject, or (b) inhibition or reduction of an increase in the amount of hypocretin and/or an increase in the amount of hypocretin neurons in a subject, which increases are caused by administration of one or more opioids. In some embodiments, the one or more opioids is provided in a therapeutically effective amount for treating, inhibiting, or reducing pain in a subject and/or the one or more hypocretin/orexin receptor antagonists is provided in a therapeutically effective amount for inhibiting or reducing the likelihood that the subject will develop an addiction to the one or more opioids. In some embodiments, the one or more opioids is selected from opioid peptides such as endorphins, enkephalins, dynorphins, and endomorphins; opium alkaloids such as codeine, morphine, thebaine, oripavine, and papaveretum; esters of morphine such as diacetylmorphine (morphine diacetate; heroin), nicomorphine (morphine dinicotinate), dipropanoylmorphine (morphine dipropionate), diacetyldihydromorphine, acetylpropionylmorphine, desomorphine, methyldesorphine, and dibenzoylmorphine; ethers of morphine such as dihydrocodeine, ethylmorphine, and heterocodeine; synthetic alkaloids such as buprenorphine, etorphine, hydrocodone, hydromorphone, oxycodone, and oxymorphone; and synthetic opioids such as anilidopiperidines (e.g., fentanyl, alphamethylfentanyl, alfentanil, sufentanil, remifentanil, carfentanyl, ohmefentanyl), phenylpiperidines (e.g., pethidine (meperidine), ketobemidone, MPPP, allylprodine, prodine, PEPAP, promedol), diphenylpropylamine derivatives (e.g., propoxyphene, dextropropoxyphene, dextromoramide, bezitramide, piritramide, methadone, dipipanone, levomethadyl acetate, difenoxin, diphenoxylate, loperamide), benzomorphan derivatives (e.g., dezocine, pentazocine, phenazocine), oripavine derivatives (e.g., buprenorphine, dihydroetorphine, etorphine), morphinan derivatives (e.g., butorphanol, nalbuphine, levorphanol, levomethorphan, racemethorphan), lefetamine, menthol, meptazinol, mitragynine, tilidine, tramadol, tapentadol, eluxadoline, AP-237, and 7-hydroxymitragynine. In some embodiments, the one or more opioids is selected from morphine, esters of morphine, ethers of morphine, synthetic opioids, and synthetic alkaloids. In some embodiments, the one or more opioids is morphine, oxymorphone, hydromorphone, fentanyl, hydrocodone, oxycodone, oxymorphone, or hydromorphone. In some embodiments, the one or more hypocretin/orexin receptor antagonists is selected from suvorexant, almorexant, EMPA, filorexant, JNJ-10397049, lemborexant, MIN-202, MK-1064, MK-8133, nemorexant, RTIOX-276, SB-334867, SB-408124, SB-649868 (CAS No. 380899-24-1), TCS-OX2-29, (3,4-dimethoxyphenoxy) alkylamino acetamides, and Compound 1 m. In some embodiments, the one or more hypocretin/orexin receptor antagonists is suvorexant. In some embodiments, the one or more opioids is selected from morphine, esters of morphine, and ethers of morphine and the one or more hypocretin/orexin receptor antagonists is suvorexant. In some embodiments, the one or more opioids is morphine and the one or more hypocretin/orexin receptor antagonists is suvorexant. In some embodiments, about 1-10 mg of the one or more opioids is provided or used. In some embodiments, about 1-10 mg of the one or more hypocretin/orexin receptor antagonists is provided or used. In some embodiments, about 1-10 mg of the one or more opioids and about 1-10 mg of the one or more hypocretin/orexin receptor antagonists are provided or used. In some embodiments, the one or more opioids and/or the one or more hypocretin/orexin receptor antagonists is used orally. In some embodiments, the one or more opioids and/or the one or more hypocretin/orexin receptor antagonists is used intravenously. In some embodiments, the medicament is an oral formulation. In some embodiments, the medicament is an intravenous formulation. In some embodiments, the medicament further comprises a pharmaceutically acceptable vehicle.

In some embodiments, the present invention provides a kit comprising one or more opioids packaged together with one or more hypocretin/orexin receptor antagonists. In some embodiments, a single dose of the one or more opioids is provided in the kits. In some embodiments, a single dose of the one or more hypocretin/orexin receptor antagonists is provided in the kits. In some embodiments, multiple doses of the one or more opioids and/or one or more hypocretin/orexin receptor antagonists are provided in the kits. In some embodiments, the one or more hypocretin/orexin receptor antagonists and/or the one or more opioids are packaged together as a pack and/or in drug delivery device, e.g., a pre-filled syringe. In some embodiments, the one or more hypocretin/orexin receptor antagonists and/or the one or more opioids are packaged together in the form of a composite pill or tablet.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 shows that there was an average 54% increase in the number of detected Hcrt neurons in human heroin addicts. FIG. 1A: Shows photomicrographs of human hypothalamic sections of a control and heroin addict. Calibration 50 μm. FIG. 1B: Shows Hcrt number in addicts vs. controls. FIG. 1C: Shows Hcrt cells in addicts vs. controls. FIG. 1D: Shows that the Hcrt size distribution was shifted downwards in both human heroin addicts and morphine treated mice with long-term opioid treatment. FIG. 1E: Illustrates the distribution and increased number of detected Hcrt cells in human addicts relative to controls. OT—optic tract, F—Fornix, MM—mammillary bodies; numbers=number of Hcrt producing neurons in section. FIG. 1F: Shows the relation between the daily morphine dose and the number of detected cells after 2 weeks in mice (F7,16=8.1, P<0.001-ANOVA). FIG. 1G: Shows that the increased number of Hcrt cells persisted for at least 4 weeks after discontinuation of morphine treatment in mice (***P<0.001, Bonferroni t test). FIG. 1H: Shows the decrease in Hcrt cell size lasted for 2 weeks (*P<0.05). FIG. 1I: Shows that colchicine, which prevents peptides from leaving the neuronal soma and thereby causes them to accumulate in the cells, increased the number of “detectable” Hcrt cells in mice by about 44%, about the size of the morphine induced increase. This is similar to the increase in detected cells produced by morphine, indicating that a large proportion of the population of neurons capable of producing Hcrt do not do so under baseline conditions, but are induced to do so by repeated doses of opioids. FIG. 1J: Shows that morphine together with colchicine does not further increase the number of Hcrt cells labelled relative to colchicine alone. Together these figures show that new neurons are not being generated by morphine. Rather neurons that did not previously generate enough Hcrt to be detected are induced to generate Hcrt. The Hcrt neurons are also shrunk by morphine to a considerable extent, with a 33% decrease in volume. FIG. 1K: Shows that colchicine does not have any effect on the number of melanin concentrating hormone (MCH) neurons, which are intermixed with Hcrt neurons. FIG. 1L: Shows the proliferation of hypothalamic microglia after morphine administration. FIG. 1M: Shows the increased average microglial volume persists for at least 26 weeks. FIG. 1N show striking morphological changes in microglia after morphine treatment (Calibration 50 μm; insert, 10 μm).

FIG. 2: Shows that blocking Hcrt receptors with the dual Hcrt receptor blocker suvorexant completely inhibited and/or prevented addiction associated increases in the number of Hcrt neurons (FIG. 2A) and the reduction in Hcrt cell size (FIG. 2B) (**P<0.001, *P<0.01, t test). This is the first description manipulation shown to affect these addiction associated changes.

FIG. 3: Shows the analgesic effect of morphine alone (6 naïve mice/group, 3 tests) and of morphine in combination with suvorexant (6 naïve mice/group, 108 tests). **P<0.01 t test. Mice are put on a device whose floor temperature increases gradually. When the mouse lifts his foot, the mouse is removed from the apparatus and the temperature evoking this pain response is recorded. As can be seen in the figure, the pain threshold is greatly increased by morphine and is further increased, to a smaller degree, by increasing morphine dose from 5 to 10 mg/kg. But suvorexant at a dose that completely inhibits and/or prevents the addiction associated increase in the number of Hcrt neurons has no significant effect on the pain threshold. In other words, the effectiveness of morphine is maintained, but the addiction associated changes in the brain are completely inhibited and/or prevented by suvorexant.

FIG. 4: Hcrt neurons in the transgenic “DTA” mice can be selectively killed by removing doxycycline from their diet. FIG. 4 shows withdrawal symptom differences between DTA-Hcrt-WT mice and DTA-Hcrt-depleted mice. FIG. 4A: When naloxone is administered to elicit withdrawal following 14 days of morphine (50 mg/kg) treatment, the DTA-Hcrt-depleted mice show greatly reduced withdrawal symptoms compared to their DTA-Hcrt-WT littermates. **P<0.01, t test. They do not show paw tremor (FIG. 4B) or rearing (FIG. 4). **P<0.01, ****P<0.0001, t test. FIG. 4D: Shows the effect of a 90% depletion of Hcrt neurons on the conditioned place aversion produced by naloxone (**P<0.002, t test), using a naloxone triggered conditioned place aversion model in the art. Thus, as in the case of blocking Hcrt receptors, removing Hcrt neruons profoundly reduces or eliminates withdrawal symptoms.

FIG. 5: Shows the anatomical changes in Hcrt related systems after morphine administration to wild type (WT) mice. There is a significant increase in Hcrt axon label intensity (FIG. 5A) and Hcrt axon length (FIG. 5B) and tyrosine hydroxylase (TH) expression (FIG. 5C) in locus coeruleus (LC) after morphine (M) (50 mg/kg for 14 days) relative to saline (S) (*P<0.05,**P<0.01, t test). FIG. 5D and FIG. 5E are confocal microscopic images showing increased Hcrt innervation of LC after longterm morphine administration. FIG. 5F maps the increase in Hcrt axon labelling in LC produced by 2 weeks of morphine administration. FIG. 5G and FIG. 5H show elevated TH levels in locus coeruleus but not in DTA-Hcrt depleted mice (FIG. 5I) given the same 14 day, 50 mg/kg treatment. FIG. 5J and FIG. 5K show that cFos expression in the LC after naloxone precipitated withdrawal was also dampened in DTA-Hcrt-completely-depleted mice (cFos is an immediate early gene whose expression in the nucleus indicates increased activity of the neuron). FIG. 5L shows significant expression of delta FosB in the accumbens of a DTA-Hcrt WT mice after 14 days of morphine, which was not seen in littermate mice with complete depletion of Hcrt neurons (DTA-Hcrt depleted). (Delta Fos B has been shown to be expressed in opioid addicted animals). (FIG. 5M, Calibration 100 μm; insert 50 aca, rostral anterior commissure, LV lateral ventricle).

FIG. 6: Shows representative EMG and EEG cycles of subjects before and after treatment with suvorexant. FIG. 6A: Shows representative EMG (top) and EEG (bottom) of Waking, NREM, and REM samples. Baseline was acquired for a 7 day control period (FIG. 6B, FIG. 6C; Control), followed by daily morphine (50 mg/kg) administration for Day 14 at ZTO (i.e., at lights on), the beginning of the normal sleep period in the subject. On Day 15, saline administration at ZTO (withdrawal) did not prevent the decrease in sleep time and increase in wakefulness during the light phase (FIG. 6B) and in the overall 24 hour period (FIG. 6C) expected during spontaneous withdrawal, but was reversed when suvorexant (30 mg/kg) was administered (only once at ZT0) (withdrawal+suvorexant). Suvorexant and Hcrt depletion were equally effective in restoring sleep (and waking) to baseline levels during withdrawal (FIG. 6B top and bottom, DTA-Hcrt-partial-depletion). For each set of bars, the left bar is “Waking”, the middle bar is “NREM”, and the right bar is “REM”.

FIG. 7: Shows the response of Hcrt neurons to single injections of morphine. FIG. 7A: Shows discharge rates as averages of five consecutive 10 second samples in each of 5 Hcrt neurons, from 3 opioid naïve rats, in each state. FIG. 7B: Shows the discharge rate of Hcrt neurons after morphine administration, with expansions below to better show EEG immediately after injection (left) and 3 hours after injection (right).

FIG. 8: Shows the reduction and/or inhibition of morphine anticipation (indicator of addiction) when morphine was given with suvorexant. FIG. 8A shows wheel running averaged over the last 12 days of the 14-day study periods for the three experimental groups after 5 mg/kg of morphine or suvorexant or both. Anticipatory running is seen in the vehicle+morphine group (dotted line) starting at ZT2 (8 AM, 2 hours after light on pulse). Running further increased after morphine injection in the vehicle+morphine group at ZT5. The anticipatory activity was completely absent in animals given suvorexant in vehicle followed by 5 mg/kg of morphine (dashed line). Suvorexant also greatly reduced running after morphine injection from ZT 5-8 (11 AM-2 PM) compared to vehicle+morphine alone, indicating, again, a major dampening of suvorexant on morphine induced motor excitation by blocking Hcrt receptors. There was no substantial “anticipatory” activity or any other activity from ZT0-12 when suvorexant in vehicle was given followed by saline injection (solid line). We ran this study with both 5 and 10 mg/kg doses of morphine with a virtually identical pattern of activity shown in both experiments (the 5 mg dose is shown in the line graphs and both 5 and 10 mg doses are shown in the bar graphs (FIG. 8B & FIG. 8C) which indicate total activity during two ZT intervals, for each of the two morphine doses used. Comparisons to vehicle-morphine condition, *P=0.05; **0.01, ***P=0.001.

### DETAILED DESCRIPTION OF THE INVENTION

The experiments disclosed herein indicate that, when opioids, e.g., morphine, are administered in combination with hypocretin/orexin receptor antagonists, their analgesic effect is maintained and the changes in the brain associated with opioid addiction are reduced or inhibited. Specifically, human heroin addicts have a great increase in the number of Hcrt producing neurons. Narcoleptic humans, who have a 90% on average loss of Hcrt producing neurons, are resistant to addiction. As shown herein, suvorexant blocks the change in the number and size of Hcrt neurons induced by chronic opioid morphine administration in subjects. The experiments herein show that suvorexant has little to no effect on the analgesia induced by morphine in subjects. The experiments herein also show that suvorexant inhibits and/or reduces morphine addiction that is expected as a result of morphine administration. Suvorexant also attenuates the motor response to morphine itself.

Therefore, the present invention provides methods and compositions for treating, inhibiting, or reducing pain in subjects with one or more opioids while reducing or inhibiting the subjects' risk of developing an addition to the one or more opioids. The methods comprise administering one or more hypocretin/orexin receptor antagonists in combination with the one or more opioids. In this context, the phrase “in combination with” means that the one or more hypocretin/orexin receptor antagonists is administered before, with, or after, i.e., within 6 hours of the administration of the one or more opioids. For example, the Hcrt antagonist could be given as early as 6 hours before opioids or as late as 6 hours after opioids. In some embodiments, the one or more hypocretin/orexin receptor antagonists is administered within 5 hours, within 4 hours, within 3 hours, within 2 hours, within 1 hour, within 30 minutes, or within 15 minutes of the administration of the one or more opioids. In some embodiments, the one or more hypocretin/orexin receptor antagonists is administered 5 hours, 4 hours, 3 hours, 2 hours, 1 hour, 30 minutes, or 15 minutes before the administration of the one or more opioids. In some embodiments, the one or more hypocretin/orexin receptor antagonists is administered 5 hours, 4 hours, 3 hours, 2 hours, 1 hour, 30 minutes, or 15 minutes after the administration of the one or more opioids. In some embodiments, the one or more hypocretin/orexin receptor antagonists is administered concurrently with the one or more opioids. In some embodiments, a single pharmaceutical composition, such as a pill, comprising one or more opioids and one or more hypocretin/orexin receptor antagonists is administered. In some embodiments, the one or more hypocretin/orexin receptor antagonists is an Hcrt receptor 1 antagonist. In some embodiments, the one or more hypocretin/orexin receptor antagonists is an Hcrt receptor 2 antagonist. In some embodiments, the one or more hypocretin/orexin receptor antagonists is suvorexant. In some embodiments, the one or more opioids is morphine. In some embodiments, the one or more hypocretin/orexin receptor antagonists is suvorexant and the one or more opioids is morphine. In some embodiments, the pain is acute pain. In some embodiments, the pain is chronic pain. In some embodiments, the subject is human. In some embodiments, a therapeutically effective amount of the one or more hypocretin/orexin receptor antagonists is administered. As used herein, a “therapeutically effective amount” of the one or more hypocretin/orexin receptor antagonists refers to an amount that inhibits or reduces an increase in the number of hypocretin (Hcrt) neurons in a subject as compared to a negative control, such as a placebo (i.e., the increase in the number of Hcrt neurons that would likely result from the administration of the one or more opioids in the absence of administration of one or more hypocretin/orexin receptor antagonists. The skilled artisan will appreciate that certain factors may influence the amount required to reduce or inhibit a given subject's risk of developing an addition to the one or more opioids. Nevertheless, effective amounts and therapeutically effective amounts may be readily determined by methods in the art.

In some embodiments, a therapeutically effective amount of the one or more hypocretin/orexin receptor antagonists ranges from about 0.01-10 mg/kg, about 0.01-3 mg/kg, about 0.01-2 mg/kg, about 0.01-1 mg/kg, or about 0.01-0.7 mg/kg body weight of the subject being treated. In some embodiments, the therapeutically effective amount of the one or more hypocretin/orexin receptor antagonists is lower than the typical amount administered for treating insomnia. In some embodiments, the therapeutically effective amount of the one or more hypocretin/orexin receptor antagonists is 0.01-0.13 mg/kg or 0.01-0.065 mg/kg body weight of the subject being treated. In some embodiments, the therapeutically effective amount of the one or more hypocretin/orexin receptor antagonists is about 0.05-0.15 mg/kg body weight of the subject being treated. In some embodiments, the therapeutically effective amount suvorexant is 0.01-0.13 mg/kg or 0.01-0.065 mg/kg body weight of the subject being treated. In some embodiments, the therapeutically effective amount of suvorexant is about 0.05-0.15 mg/kg body weight of the subject being treated.

In some embodiments, a therapeutically effective amount of the one or more opioids is administered. As used herein, a “therapeutically effective amount” of the one or more opioids refers to an amount that inhibits or reduces pain in a subject as compared to a negative control, such as a placebo. The skilled artisan will appreciate that certain factors may influence the amount required to reduce or inhibit pain in a given subject. Nevertheless, effective amounts and therapeutically effective amounts may be readily determined by methods in the art. In some embodiments, the therapeutically effective amount of a given opioid is the dose recommended by the manufacturer of the given opioid and/or the dose approved by the U.S. FDA for the given opioid.

In some embodiments, a therapeutically effective amount of the one or more opioids ranges from about 0.001-0.5 mg/kg, about 0.001-0.4 mg/kg, about 0.001-0.3 mg/kg, about 0.001-0.2 mg/kg, about 0.001-0.1 mg/kg body weight of the subject for parenteral administration. In some embodiments where the opioid is morphine, the therapeutically effective amount ranges from about 0.10-0.20 mg/kg, about 0.10-0.19 mg/kg, about 0.10-0.18 mg/kg, about 0.10-0.17 mg/kg, about 0.10-0.16 mg/kg, more preferably about 0.14-0.18 mg/kg, even more preferably about 0.15-0.17 mg/kg, and most preferably about 0.16 mg/kg body weight of the subject for parenteral administration. In some embodiments where the opioid is oxymorphone, the therapeutically effective amount ranges from about 0.004-0.020 mg/kg, about 0.005-0.019 mg/kg, about 0.006-0.018 mg/kg, about 0.007-0.017 mg/kg, about 0.008-0.016 mg/kg, about 0.009-0.015 mg/kg, about 0.010-0.014 mg/kg, about 0.011-0.015 mg/kg, or about 0.012-0.014 mg/kg for parenteral administration. In some embodiments where the opioid is hydromorphone, the therapeutically effective amount ranges from about 0.001-0.10 mg/kg, about 0.005-0.09 mg/kg, about 0.01-0.08 mg/kg, about 0.02-0.07 mg/kg, about 0.03-0.06 mg/kg, about 0.04-0.05 mg/kg, or about 0.04 mg/kg body weight of the subject for parenteral administration. In some embodiments where the opioid is fentanyl, the therapeutically effective amount ranges from about 0.05-2.5 mcg/kg, about 0.06-1.4 mcg/kg, about 0.07-2.3 mcg/kg, about 0.08-2.2 mcg/kg, about 0.09-2.1 mcg/kg, about 1.1-2.0 mcg/kg, about 1.2-1.9 mcg/kg, about 1.3-1.8 mcg/kg, about 1.4-1.7 mcg/kg, or about 1.5-1.6 mcg/kg body weight of the subject for parenteral administration.

Therapeutically effective amounts for oral administration may be up to about 10-fold higher. In some embodiments, a therapeutically effective amount of the one or more opioids ranges from about 0.01-1.0 mg/kg, about 0.05-0.90 mg/kg, about 0.06-0.80 mg/kg, about 0.07-0.70 mg/kg, about 0.08-0.60 mg/kg, about 0.09-0.50 mg/kg, or about 0.10-0.40 mg/kg body weight of the subject for oral administration. In some embodiments where the opioid is morphine or hydrocodone, the therapeutically effective amount ranges from about 0.01-1.0 mg/kg, about 0.25-0.75 mg/kg, about 0.30-0.50 mg/kg, or about 0.40 mg/kg body weight of the subject for oral administration. In some embodiments where the opioid is oxycodone, the therapeutically effective amount ranges from about 0.05-0.50 mg/kg, about 0.10-0.40 mg/kg, about 0.20-0.30 mg/kg, or about 0.27 mg/kg body weight of the subject for oral administration. In some embodiments where the opioid is oxymorphone, the therapeutically effective amount ranges from about 0.01-0.25 mg/kg, about 0.05-0.20 mg/kg, about 0.10-0.15 mg/kg, or about 0.13 mg/kg body weight of the subject for oral administration. In some embodiments where the opioid is hydromorphone, the therapeutically effective amount ranges from about 0.01-0.20 mg/kg, about 0.05-0.15 mg/kg, or about 0.1 mg/kg body weight of the subject for oral administration.

The one or more hypocretin/orexin receptor antagonists and the one or more opioids are preferably administered to the subject in the form of a composite pharmaceutical composition—a pharmaceutical composition comprising one or more hypocretin/orexin receptor antagonists in a therapeutically effective amount, one or more opioids in a therapeutically effective amount, and a pharmaceutically acceptable vehicle. The pharmaceutical compositions may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

In some embodiments, compositions comprising one or more opioids and one or more hypocretin/orexin receptor antagonists are provided. In some embodiments, the compositions for parenteral administration comprise about 0.005-20 mg of the one or more opioids. In some embodiments, compositions for parenteral administration comprise about 1-25 mg, about 5-20 mg, about 10-15 mg, or about 12 mg of morphine. In some embodiments, compositions for parenteral administration comprise about 0.005-2.5 mg, about 0.05-2.0 mg, about 0.5-1.5 mg, or about 1 mg of oxymorphone. In some embodiments, compositions for parenteral administration comprise about 0.1-10 mg, about 1-5 mg, about 2-4 mg, or about 3 mg of hydromorphone. In some embodiments, compositions for parenteral administration comprise about 10-250 mcg, about 50-200 mcg, about 100-150 mcg, or about 120 mcg of fentanyl. In some embodiments, the compositions for oral administration comprise about 1-50 mg of the one or more opioids. In some embodiments, compositions for oral administration comprise about 5-50 mg, about 10-40 mg, about 20-35 mg, or about 30 mg of morphine or hydrocodone. In some embodiments, compositions for oral administration comprise about 1-40 mg, about 5-35 mg, about 10-30 mg, or about 20 mg of oxycodone. In some embodiments, compositions for oral administration comprise about 0.5-25 mg, about 1-20 mg, about 5-15 mg, or about 10 mg of oxymorphone. In some embodiments, compositions for oral administration comprise about 0.1-20 mg, about 1-15 mg, about 5-10 mg, or about 7.5 mg of hydromorphone.

In some embodiments, the compositions comprise about 1-40 mg, about 1-25 mg, about 1-20 mg, about 1-15 mg, about 1-10 mg, about 1-5 mg, or about 5 mg of the one or more hypocretin/orexin receptor antagonists. In some embodiments, the compositions comprise about 5-30 mg, about 5-25 mg, about 5-20 mg, about 5-15 mg, about 5-10 mg, or about 5 mg of the one or more hypocretin/orexin receptor antagonists. In some embodiments, the compositions comprise about 10-30 mg, about 10-25 mg, about 10-20 mg, about 10-15 mg, or about 10 mg of the one or more hypocretin/orexin receptor antagonists. In some embodiments, the compositions comprise about 15-30 mg, about 15-25 mg, about 15-20 mg, or about 15 mg of the one or more hypocretin/orexin receptor antagonists. In some embodiments, the compositions comprise about 20-30 mg, about 20-25 mg, or about 20 mg of the one or more hypocretin/orexin receptor antagonists. In some embodiments, the compositions comprise about 0.1 mg to less than 10 mg of the one or more hypocretin/orexin receptor antagonists. In some embodiments, the compositions comprise about 0.1 mg to less than 5 mg of the one or more hypocretin/orexin receptor antagonists. In some embodiments, the compositions comprise about 0.1 mg to less than 10 mg of suvorexant. In some embodiments, the compositions comprise about 0.1 mg to less than 5 mg of suvorexant.

In some embodiments, the weight to weight ratio of the one or more opioids to the one or more hypocretin/orexin receptor antagonists in the compositions is about 0.01:1, about 0.05:1, about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, about 1:0.1, about 1:0.05, or about 1:0.01. In some embodiments, the weight to weight ratio of morphine to suvorexant in the compositions is 1:0.15, 1:0.3, 1:0.4, or 1:0.8 (morphine:suvorexant).

The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier. In some embodiments, the pharmaceutical composition is a composite of one or more opioids such as morphine, buprenorphine, methadone, and the like, and one or more hypocretin/orexin receptor antagonists, such as dual Hcrt receptor antagonists, Hcrt 1 antagonists, and/or Hcrt 2 antagonists in appropriate dosages.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intrarectal, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular hypocretin/orexin receptor antagonist and/or the particular opioid used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, Md.

The pharmaceutical compositions may be provided in dosage unit forms. As used herein, a “dosage unit form” refers to physically discrete units suited as unitary dosages for the subject to be treated; each unit containing a predetermined quantity of the one or more hypocretin/orexin receptor antagonist calculated to produce the desired therapeutic effect in association with the required pharmaceutically acceptable carrier. The specification for the dosage unit forms of the invention are dictated by and directly dependent on the unique characteristics of the given hypocretin/orexin receptor antagonist and desired therapeutic effect to be achieved, and the limitations inherent in the art of compounding such an active compound for the treatment of individuals.

Toxicity and therapeutic efficacy of hypocretin/orexin receptor antagonists according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. hypocretin/orexin receptor antagonists which exhibit large therapeutic indices are preferred. While hypocretin/orexin receptor antagonists that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more hypocretin/orexin receptor antagonists can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

### Kits

In some embodiments, the present invention provides kits comprising one or more hypocretin/orexin receptor antagonists packaged together with one or more opioids for preventing, inhibiting, reducing, or treating pain in a subject. In some embodiments, the one or more hypocretin/orexin receptor antagonists and/or the one or more opioids are packaged together as a pack and/or in drug delivery device, e.g., a pre-filled syringe. In some embodiments, the one or more hypocretin/orexin receptor antagonists and/or the one or more opioids are packaged together in the form of a composite pill or tablet.

In some embodiments, the kits optionally include an identifying description or label or instructions relating to its use. In some embodiments, the kits include information prescribed by a governmental agency that regulates the manufacture, use, or sale of compounds and compositions as contemplated herein.

The following examples are intended to illustrate but not to limit the invention.

### EXAMPLES

### Addiction-Linked Increases in the Number of Hcrt Producing Neurons Caused by Opioids is Inhibited or Reduced by Blocking Hcrt Receptors

The administration of an Hcrt receptor antagonist suvorexant can produce a complete suppression of the opiate induced, addiction-associated, increase in the number, and decrease in the size, of detectable Hcrt neurons, as well as reducing, inhibiting, and/or preventing the opioid anticipation characteristic of addiction.

At 50 mg/kg doses of morphine (the dose that produces the maximal anatomical change in Hcrt neurons in mice), suvorexant administered up to 1 hour before or 1 hour after morphine, blocks the addiction associated increase in the number, and the shrinkage in the size, of Hcrt cells produced by daily morphine alone. Hcrt R1 blockade (with SB-334867 or ACT-335827), also reduces, inhibits, and/or prevents the changes in Hcrt neurons produced by chronic morphine administration. Suvorexant by itself does not affect Hcrt cell numbers.

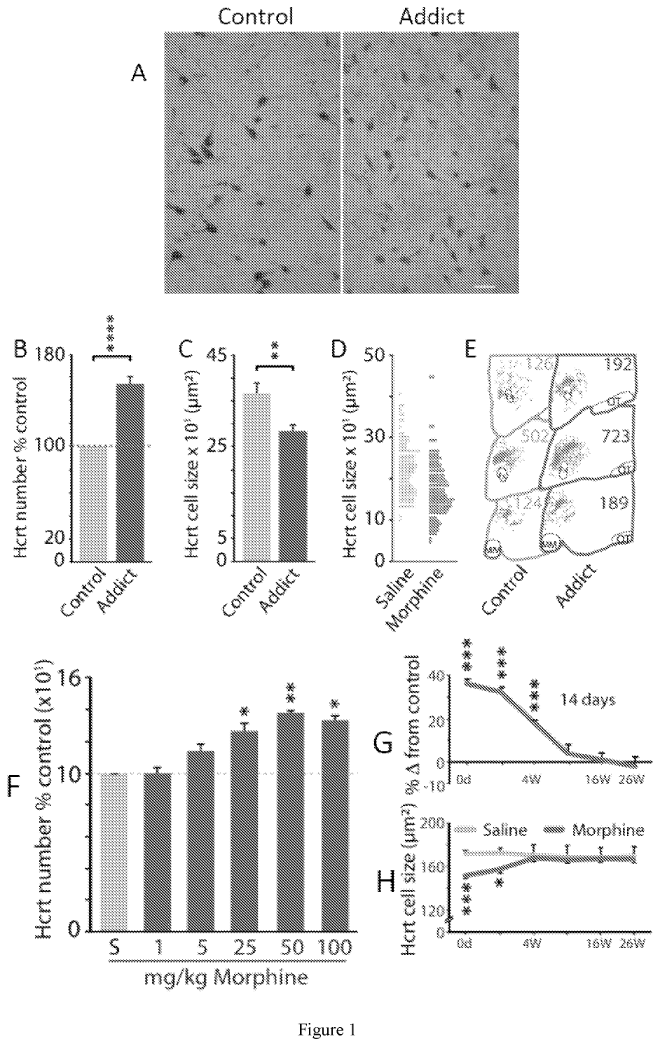

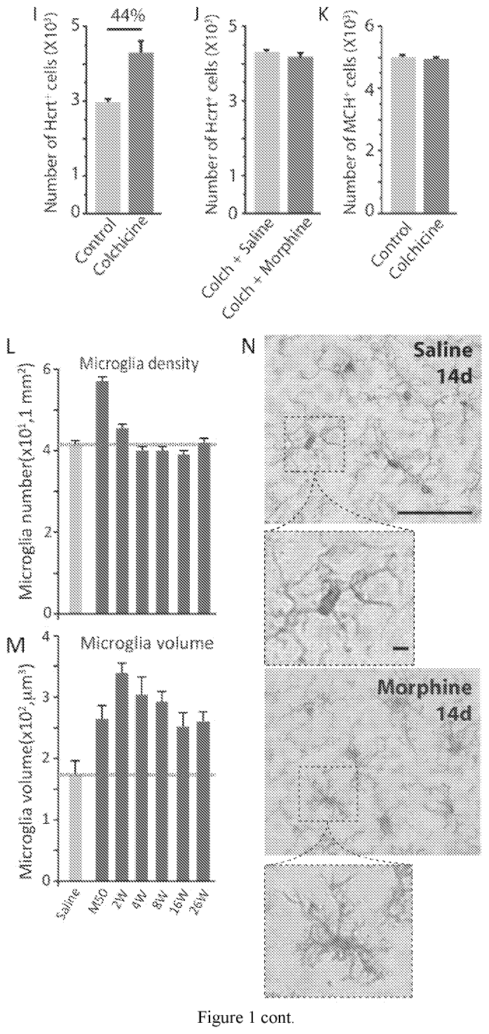

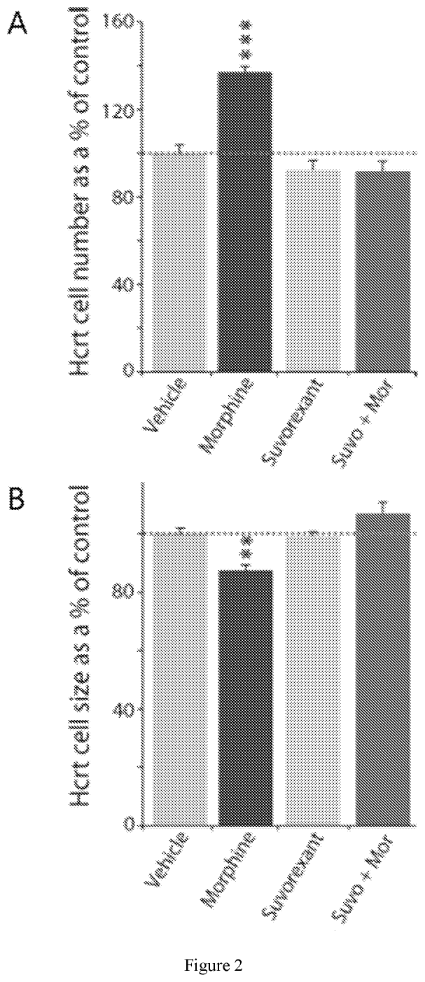

As shown in FIG. 1, long-term administration of heroin in humans or addictive levels of morphine in mice, and cocaine in rats, produce an increase in the number of detected Hcrt neurons, activating Hcrt production in a large subpopulation of hypothalamic neurons that do not normally produce detectable levels of the peptide. Because of the role of Hcrt neurons in pleasure seen in rodents, cats, dogs, and humans we wondered if blocking Hcrt receptors might affect the addiction linked increase in the number of detectable Hcrt neurons. Blocking Hcrt receptors produced a very large effect, with physiological doses of suvorexant, 30 mg/kg (in 0.5% methyl cellulose vehicle P.O. administered 60 min before each daily morphine injection SQ for 2 weeks), completely inhibiting or preventing the addiction associated increase in the number of Hcrt neurons (FIG. 2A) and the reduction in Hcrt cell size (FIG. 2B) (**P<0.001, *P<0.01, t test).

At the dose used, even though given in the light period (the normal sleep period for mice) no sleep was observed, likely due to the arousing quality of opioids in mice and rats. Therefore, even though suvorexant is a “sleeping pill,” sleep did not mediate the observed effect of this drug on Hcrt cell number and size seen in FIG. 2.

### Hcrt Receptor Blockade Does Not Impact Analgesia Effects of Morphine

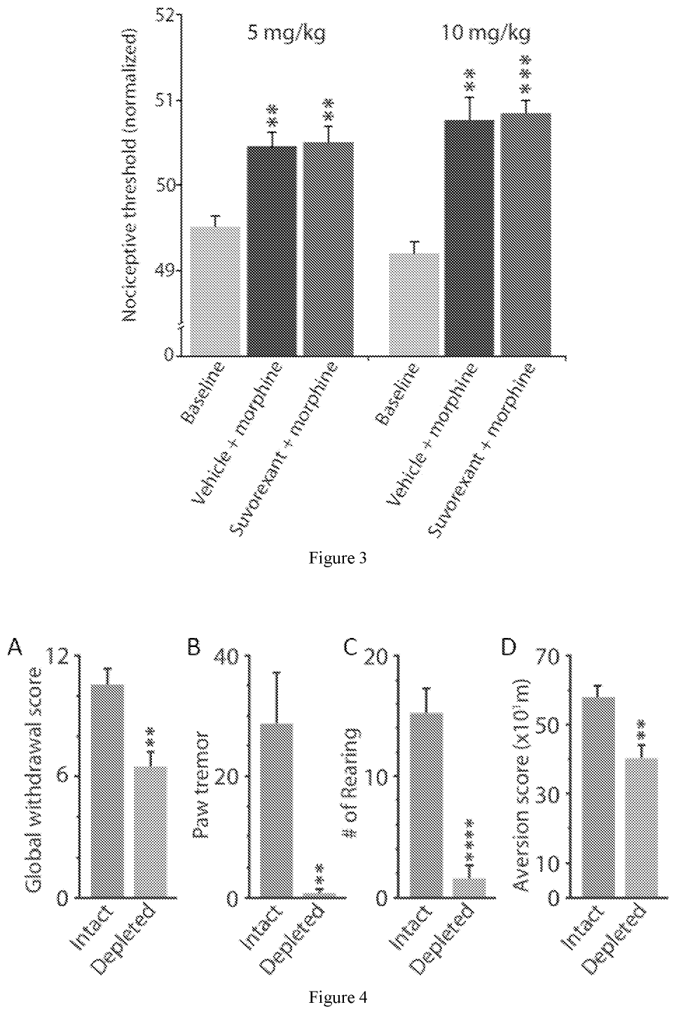

Neither Hcrt receptor blockade nor deletion of Hcrt neurons using the DTA transgenic mouse substantially reduced the analgesic effects of morphine. Analgesia effects were assessed using the pain response threshold and latency in the thermal nociceptive (FIG. 3) and formalin tests in the art across a wide range of doses.

The effect of suvorexant on the pain threshold was tested using an IITC PE34 Incremental Thermal Nociceptive Threshold Analgesia Meter, which raises the surface temperature at 6° C./min until the mouse licks or shakes a hind limb, or jumps, at which point the stop switch is pressed by the investigator (who is always bind to the treatment condition and the mouse is removed from the apparatus. For latency, the plate is set to 55° C. and the response delay recorded. Baseline threshold or latency is established on 3 consecutive days, with 3 tests/day. During drug treatments, 2 tests, a pre-drug and a 1-hour post-drug, are done daily. The animal was checked for any skin inflammation or lesion and removed immediately from the experiment for treatment if either occurs. The formalin test was a supplemental approach to measuring morphine analgesia, analogous to clinical situations in which C-fiber function is implicated. It was conducted just once on each mouse 24 hours after the last thermal test by injecting 20 μl of 4% formalin or saline subcutaneously and recording the time spent licking, or lifting the injected hind paw over 45 minutes.

The analgesic effect of morphine alone can be seen in FIG. 3 left (P<0.01, t test, comparing baseline (vehicle) to the 5 mg/kg morphine+vehicle effect on the paw raising response to floor heating. The average analgesic effect (n=6/group, 3 tests) to heat is not significantly diminished by the same 30 mg/kg oral (by gavage) dose of suvorexant+vehicle FIG. 3 right (P<0.01 t test) that reduced, inhibited, and/or prevented the addiction associated increase in Hcrt cell number seen in FIG. 2 (with 5 mg/kg doses of morphine, well below the level at which nonspecific effects including the Straub tail occur). The analgesic effect elicited by morphine with vehicle and morphine with suvorexant did not significantly differ (<0.6° C. difference). This is based on morphine/suvorexant in 6 naive mice/group, a total of 108 tests. The results shown in FIG. 2 and FIG. 3 indicate that administration of an Hcrt receptor inhibitor such as suvorexant before or with opioid administration reduces, inhibits, and/or prevents opioid “addiction” associated changes in Hcrt neurons without significantly diminishing the analgesic effect of the opioid.

### Hcrt Neuron Removal or Hcrt Receptor Blockade Reduces or Inhibits Withdrawal Symptoms and Morphine Effects on Locus Coeruleus and Histamine Neurons

The increase in the detected number of histamine neurons and of tyrosine hydroxylase expression in the locus coeruleus produced by opioids is reduced, inhibited, or prevented by the lowest doses of suvorexant effective in inhibiting or preventing the increase in Hcrt cell number and decrease in their size produced by chronic administration of morphine.

### Removal of Hcrt Neurons (or Blockade of Hcrt Receptors) Prevents or Greatly Reduces Opioid Withdrawal Symptoms

DTA-Hcrt-depleted and DTA-Hcrt-WT mice were given a once per day dose of morphine 50 mg/kg, SC for 14 days. Two hours after the last injection, naloxone (2 mg/kg, SC) was administered and withdrawal symptoms assessed. The mice were videotaped and locomotion, jumping, backward stepping, rearing, paw tremor, teeth chattering, grooming, behavioral arrest, defecation, urination, wet dog shake, ptosis, diarrhea, body tremor, and piloerection was quantified. A global withdrawal score was calculated using methods in the art.

FIG. 4A-FIG. 4C show major differences between DTA-Hcrt-WT mice (green- no loss of Hcrt neurons) and DTA-Hcrt-depleted mice, i.e., mice in which the Hcrt neurons have been removed (complete ablation-orange) in these initial studies. When naloxone was administered to elicit withdrawal following 14 days of morphine (50 mg/kg) treatment, the DTA-Hcrt-depleted mice showed greatly reduced withdrawal symptoms compared to their DTA-Hcrt-WT littermates. Their overall global score on withdrawal behavior was significantly reduced (FIG. 4A, **P<0.01, t test). They did not show paw tremor or rearing (FIG. 4B, FIG. 4C; **P<0.01, ****P<0.0001, t test). FIG. 4D shows the effect of a 90% depletion of Hcrt neurons on the conditioned place aversion produced by naloxone (**P<0.002, t test), using a validated naloxone triggered conditioned place aversion model in the art. These results indicate that blocking Hcrt receptors or eliminating Hcrt neurons may eliminate withdrawal symptoms.

These data indicate that the Hcrt system has a major role in both addiction and withdrawal. At one extreme, it appears that if these neurons are eliminated, withdrawal symptoms are greatly reduced, as they are in narcoleptic humans (who, on average have a 90% loss of Hcrt neurons) and DTA-Hcrt-depleted mice (above). As shown in FIG. 2, suvorexant administration with morphine prevents or inhibits the changes in the number and size of Hcrt producing neurons that characterize opioid and cocaine addiction. Suvorexant alone has no effect on the number and size of Hcrt neurons. The tight correlation of Hcrt cell number increase with addiction in mice and opioid use disorder in humans, (FIG. 1-FIG. 3) indicates that it is possible to separate the analgesic and addictive effects of opioids in subjects, by administering an Hcrt receptor inhibitor such as suvorexant before or with the opioids.

Opiate addiction increases the number of detectable Hcrt neurons in both humans and mice. As shown in FIG. 1I and FIG. 1J, the increase in Hcrt neurons is not due to neurogenesis. Rather the increase is due to increased amounts of Hcrt being generated by neurons that are capable of producing detectable levels of Hcrt, but do not do so in non-addict humans or in mice under our baseline conditions.

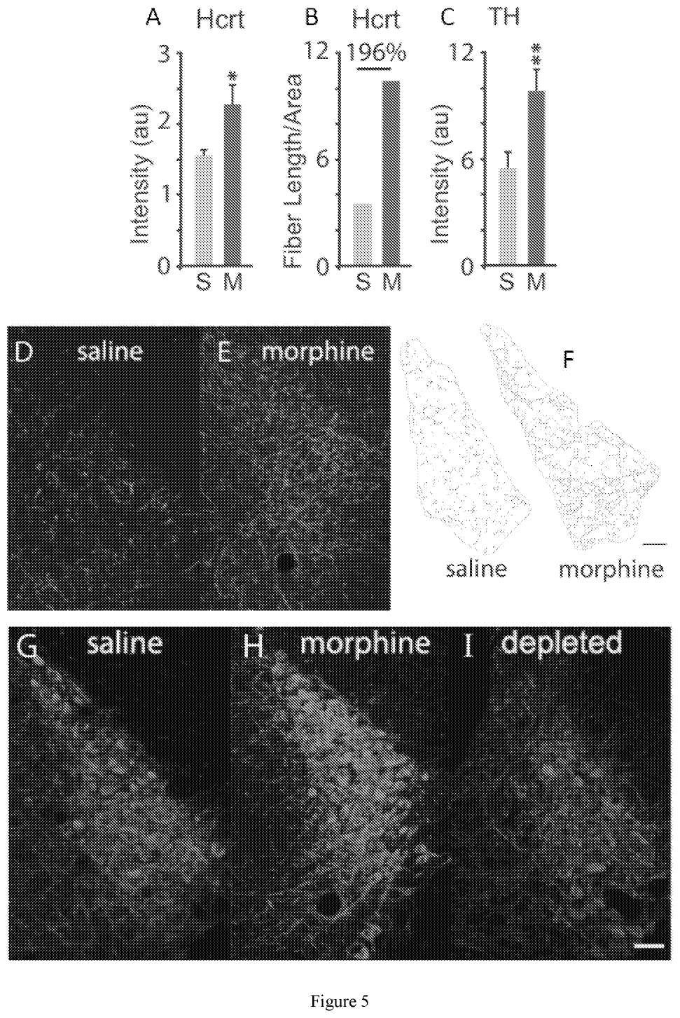

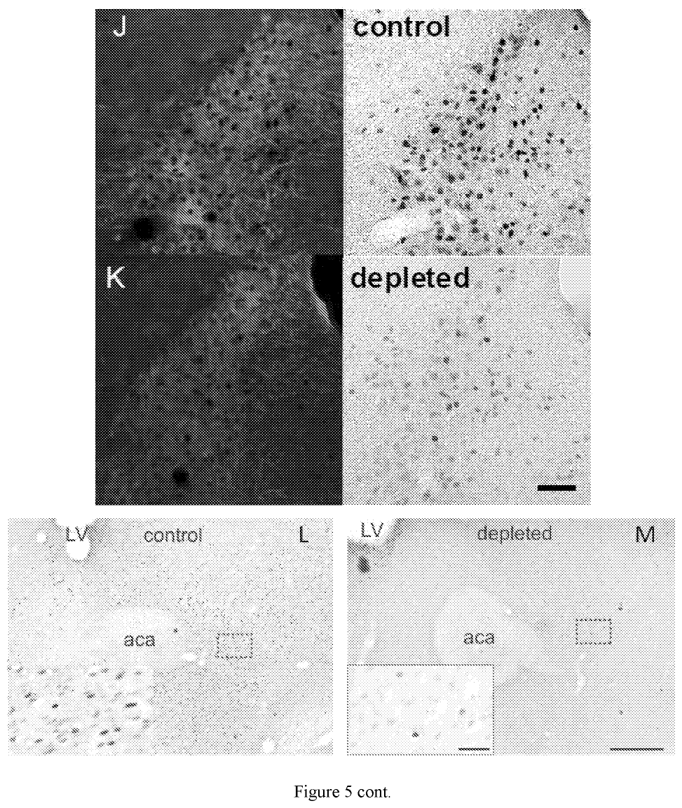

This data (FIG. 5) shows anatomical changes in Hcrt related systems after morphine administration. There is a significant increase in Hcrt axon label intensity (FIG. 5A) and Hcrt axon length (FIG. 5B) and TH expression (FIG. 5C) in LC after morphine (M) (50 mg/kg for 14 days) relative to saline (S) (*P<0.05,**P<0.01, t test). Confocal images in FIG. 5D and FIG. 5E show examples of increased Hcrt innervation of LC. FIG. 5F maps the increase in Hcrt axon labelling in LC produced by 2 weeks of morphine administration. (Hcrt axons are absent in the LC in the DTA-Hcrt-neuron depleted mice.) Elevated TH levels are seen in locus coeruleus (FIG. 5G, FIG. 5H) but not in DTA-Hcrt depleted mice given the same Day 14, 50 mg/kg treatment (FIG. 5I), indicating that the morphine induced increase in LC TH level is completely dependent on Hcrt neurons. cFos expression in the LC after naloxone precipitated withdrawal, was also dampened in DTA-Hcrt-completely-depleted mice (FIG. 5J, FIG. 5K). The role of the LC in withdrawal appears to be indirect, reflecting the activity of its inputs, including habenula, thalamic paraventricular nucleus, accumbens and the dopamine system. All these structures are also linked to the symptoms of opioid administration and withdrawal.

In another set of experiments, the expression of delta Fos B, a marker of chronic neuronal activation related to the development of addiction was evaluated. FIG. 5L shows significant expression of delta FosB in the accumbens of a DTA-Hcrt WT mice after 14 days of morphine. This is not seen in littermate mice with complete depletion of Hcrt neurons (DTA-Hcrt depleted) (FIG. 5M, Cal. 100 μ; insert 50 μm, aca, rostral anterior commissure, LV lateral ventricle).

### Effect of Hcrt Receptor Blockade Prior to Morphine Administration on Sleep-Waking Cycles

The experiments herein suggest that (a) suvorexant inhibits and/or prevents increases in the number and decrease in size of Hcrt producing neurons caused by morphine and greatly reduces the immediate sleep disruption induced by morphine in mice, (b) suvorexant or Hcrt R1 blockade doses effective in reversing addiction associated changes in Hcrt neurons normalizes the EEG power spectrum across the sleep wake cycle, and (c) suvorexant administration during a 2 week period of daily morphine administration reduces or prevents the insomnia for, at least, the 2 weeks after cessation of morphine administration.

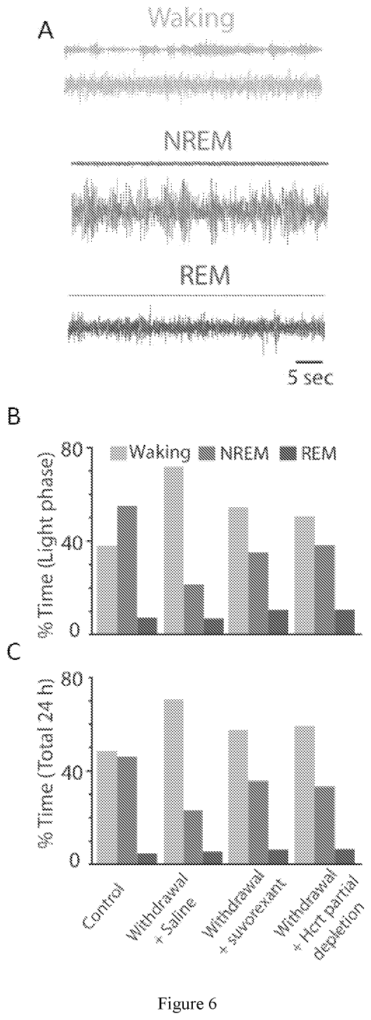

Pilot studies on opiate effects on sleep on two DTA-Hcrt-WT mice (continuously monitoring the electromyogram (EMG) and electroencephalogram (EEG) using telemetry with a DSI telemetry system) were conducted. FIG. 6A shows representative EMG (top) and EEG (bottom) samples. Baseline was acquired for a 7 day control period (FIG. 6B, FIG. 6C; Control), followed by daily morphine (50 mg/kg) administration for 14 days at ZT0. On Day 15, saline administration at ZT0 (withdrawal saline) did not prevent the decrease in sleep time and increase in wakefulness during the light phase (FIG. 6B) and in the overall 24 hour period (FIG. 6C) expected during spontaneous withdrawal. This was reversed when suvorexant (30 mg/kg) was administered (only once at ZT0) (FIG. 6B, FIG. 6C; withdrawal suvorexant). In a second pilot study, morphine was administered for 17 days. On Day 14, DOX food was replaced with regular chow for a 3-day period and then DOX food was restored to produce a 40-50% depletion of Hcrt neurons. Suvorexant was as effective as Hcrt neuron depletion in restoring sleep (and waking) to baseline levels (FIG. 6B top and bottom, DTA-Hcrt-partial-depletion).

### Effect of Hcrt Receptor Blockade on the Activity of Hcrt Neurons After Opiate Administration

The studies herein suggest that Hcrt receptor inhibitors such as suvorexant reduces, inhibits, and/or prevents anatomical changes related to opioid addiction and thereby reduces, inhibits, and/or prevents opioid addiction.

There was an average 54% increase in the number of detected Hcrt neurons in human heroin addicts (n=5) relative to controls (n=7, ***P=0.0009, t=8.89). FIG. 1A: Photomicrographs of human hypothalamic sections of a control (left) and heroin addict (right): calibration 50 μm. Note that there are more Hcrt stained neurons in the addicts. FIG. 1B: Hcrt number in addicts vs. controls. The Hcrt count was independent of the antibodies employed. FIG. 1C: Hcrt cells in human heroin addicts were 22% smaller in cross sectional area in the addicts, with a 32% decrease in volume (**P<0.001, t=2.78) and consequently somewhat less intense staining (FIG. 1A). The entire Hcrt size distribution was shifted downwards in both human heroin addicts and morphine treated mice (FIG. 1D) with long-term opioid treatment. The number and size of hypothalamic melanin concentrating hormone neurons, intermixed with Hcrt neurons were unaffected by opioids.

FIG. 1E illustrates the distribution and increased number of Hcrt cells in human addicts relative to controls. OT—optic tract, F—Fornix, MM—mammillary bodies; numbers=number of Hcrt neurons in section; FIG. 1F: shows the relation between the daily morphine dose and the number of detected cells after 2 weeks in mice (F7,16 =8.1, P<0.001-ANOVA). The maximum increase in Hcrt numbers was seen with daily injection of 50 mg/kg morphine. Morphine had to be given for at least 2 weeks to produce a significant change in the number of Hcrt cells in mice. The opioid antagonist naltrexone given alone on the same dose schedule as morphine did not change the number of Hcrt neurons (data not shown). The increase in the number of detected Hcrt cells was not due to neurogenesis. Both BrdU and doublecortin labelling indicated that no new neurons were produced by morphine, indicating that a portion of the population of Hcrt neurons does not produce detectable levels of Hcrt under baseline conditions, but that morphine elevates Hcrt level in these neurons. A significant elevation of brain Hcrt level after chronic opiate administration was seen in western blots. The increased number of Hcrt cells persisted for at least 4 weeks after discontinuation of morphine treatment in mice (FIG. 1G: ***P<0.001, Bonferroni t test). The decrease in Hcrt cell size lasted for 2 weeks (FIG. 1H: *P<0.05).

The data suggests that the increase in neurons producing detectable levels of Hcrt may last much longer in human addicts. In a further study, the issue of where the “newly visible” Hcrt cells are coming from was explored by giving colchicine to drug naive mice. Injection of colchicine into the lateral ventricle blocks axonal transport, thereby causing peptide to accumulate in the cell body. This manipulation increased the number of “detectable” Hcrt cells in mice by about 44% (FIG. 1I), similar to the amount of increase seen in mice after morphine, i.e., as many as 44% of the neurons capable of producing Hcrt in mice do not produce it at detectable levels under “baseline” conditions. FIG. 1J shows that morphine together with colchicine does not further increase the number of cells labelled relative to colchicine alone. Together, FIG. 1I and FIG. 1J show that there is a ceiling to morphine effects on Hcrt number, implying a fixed number of cells capable of producing Hcrt, with 44% of the control number of these cells (in mice) and at least 54% of control (in humans) not producing detectable levels of Hcrt under baseline conditions. FIG. 1K shows that colchicine does not have any effect on the number of melanin concentrating hormone (MCH) neurons, a peptide of similar size, whose neurons are intermixed with Hcrt cells. FIG. 1L shows the proliferation of hypothalamic microglia after morphine administration. The increased number returns to baseline by 4 weeks after the cessation of morphine. But the increased average microglial volume persists for at least 26 weeks (FIG. 1M). Microglia show striking morphological changes after morphine treatment (FIG. 1N, top saline; bottom morphine, Cal. 50 μm; insert, 10 μm). The data also suggests that the increase in neurons producing detectable levels of Hcrt may last much longer in human addicts.

Mapping and measurement were done on coded tissue, so that the person quantifying the data was always blind to condition. Mice to be compared were sacrificed and processed together. Under 40× magnification, labeled somata were identified, counted and mapped onto reconstructions of each section. Hcrt cells having a visible nucleus were outlined to allow analysis by the MicroBrightField Nucleator program, which produces a number of morphometric measures from a single tracing of the somata including area, roundness, convexity, aspect ratio and shape factor. Principal component analysis was used to find the parameters that discriminate between cells in morphine-administered mice and control mice. Hcrt labeled axons were counted and plotted using the confocal image stack (see FIG. 5). Sampling parameters were adjusted so that the coefficient of error was 0.05. In addition to quantitative assessments, nuclear fragmentation, chromatolysis, inclusions, varicosities and other abnormalities were examined and Hcrt levels in the CSF were measured.

For the diaminobenzidine tetrahydrochloride (DAB) method, tissue was pre-treated with H₂O₂ (0.3%), followed by blocking serum, primary antibody, the corresponding biotinylated secondary antibody in PBST (Jackson ImmunoResearch, West Grove, Pa., USA), standard ABC (Vector Laboratories) and developed by immersion in 0.02% DAB and 0.03% hydrogen peroxide PBS for 8 minutes. Rabbit anti-Hcrt-1 (H-003-30, Phoenix Pharmaceuticals, USA, 1:2000). For cFos and FosB visualization the DAB nickel-enhanced method is used. Methods in the art were used to distinguish FosB and delta FosB labelling. Rabbit anti-Hcrt-1 (H-003-30, Phoenix Pharmaceuticals, USA, 1:2000,), rabbit anti-cFos (ABE457, Chemicon, USA, 1:5000), FosB, goat anti-FosB (AF2214, Novus Biological, USA, 1:10000), guinea pig anti-prodynorphin (AB 5519, EMD Millipore, Darmstadt, Germany, 1:1000), and chicken anti-GFP (ab13970 Abcam, USA) were used. Identification of noradrenergic and dopaminergic neurons was performed by sheep anti-TH (tyrosine hydroxylase) (ab113, Abcam, USA, 1:2000) (and glutamatergic/Hcrt neurons by vesicular glutamate transporter-2 staining (guinea pig anti-VGlut2, AB2251-I, Millipore, USA, 1:1000)). The number and distribution of immunolabeled neurons was determined in every third section throughout the region of interest. For brightfield visualization, a Nikon Eclipse 80i microscope with three-axis motorized stage, video camera, Neurolucida interface, and Stereo Investigator software (Micro-BrightField) was used. For immunofluorescence visualization, a confocal microscope (LSM710, Carl Zeiss GmbH) was used. Western blots were used to determine peptide levels.

The data were collected at the same circadian time in drug-treated and control animals (between ZT 6 and 8). A more extensive study of circadian variations in the number and morphology of Hcrt cells may be undertaken on drug-treated and control mice using methods in the art.

Numbers of animals were determined in each group according to the formula: n=16*[std dev/(population mean−hypothesized experimental group mean)]2 with p set at 0.05 and power set at 80%.

Microwire recording techniques in the art can record single neurons for periods of weeks to months at a millisecond level of resolution of the physiological substrates of addiction and allow interspike interval histograms, autocorrelograms and action potential waveform analysis indicative of changes in ion flux, conduction velocity and long-term behavioral data to be measured. The response of Hcrt neurons changes to daily doses of morphine over a period of at least 2 weeks, a duration that elevates the number and decreases the size of Hcrt neurons (FIG. 1F, FIG. 1G), can be examined with and without concurrent Hcrt receptor blockade using microwire recording techniques in the art.

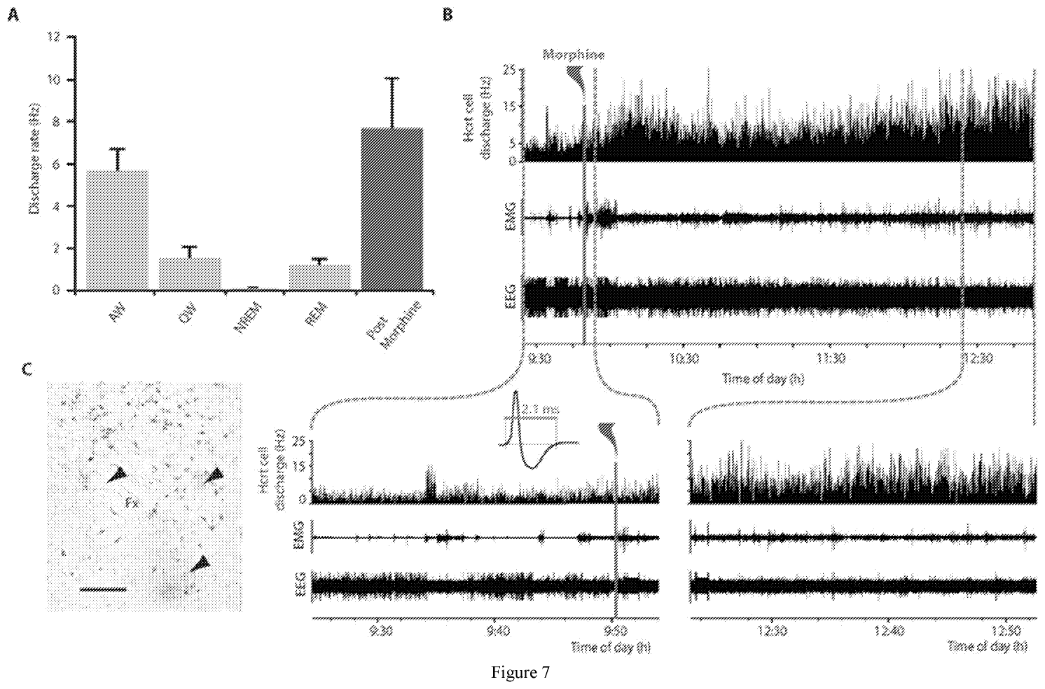

FIG. 7 shows the response of Hcrt neurons to single injections of morphine. FIG. 7A: rates are averages of five consecutive 10 second samples in each of 5 Hcrt neurons, from 3 opioid naive rats, in each state. Every injection produced greatly increased discharge. FIG. 7B: shows the discharge rate of Hcrt neurons after morphine administration, with expansions below to better show EEG immediately after injection (left) and 3 hours after injection (right). The increased discharge rate in Hcrt neurons lasted 3 or more hours after injection of morphine. Inset shows the characteristic long duration average waveform of an “opioid naive” Hcrt neuron. The data indicates that morphine injections produce a striking increase in burst discharge visible in the interspike interval histograms and even more clearly in the autocorrelograms. These were taken after a single morphine administration (FIG. 7C).

### Reduction in Morphine Anticipation when Administered with Suvorexant

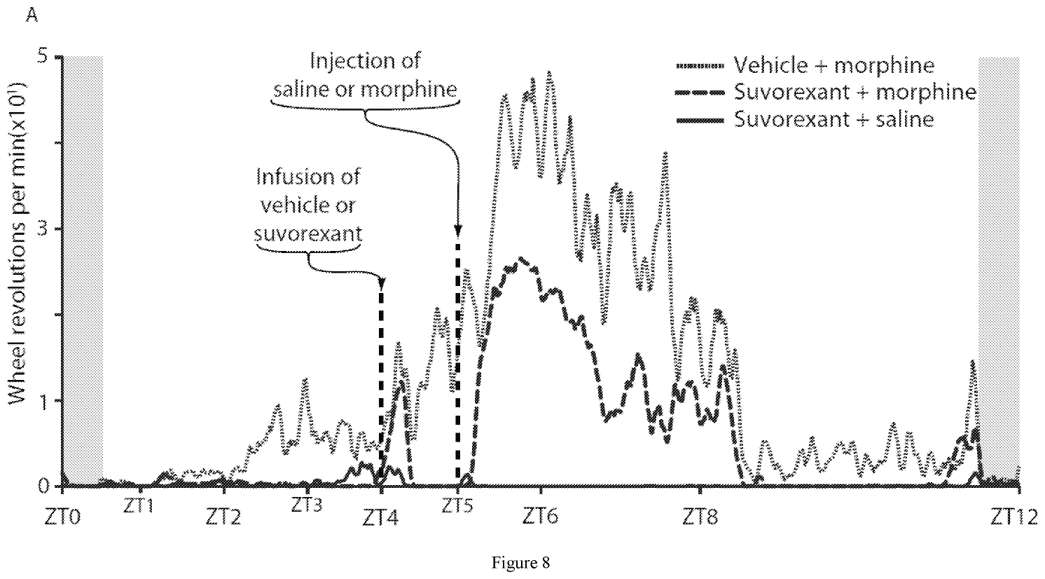

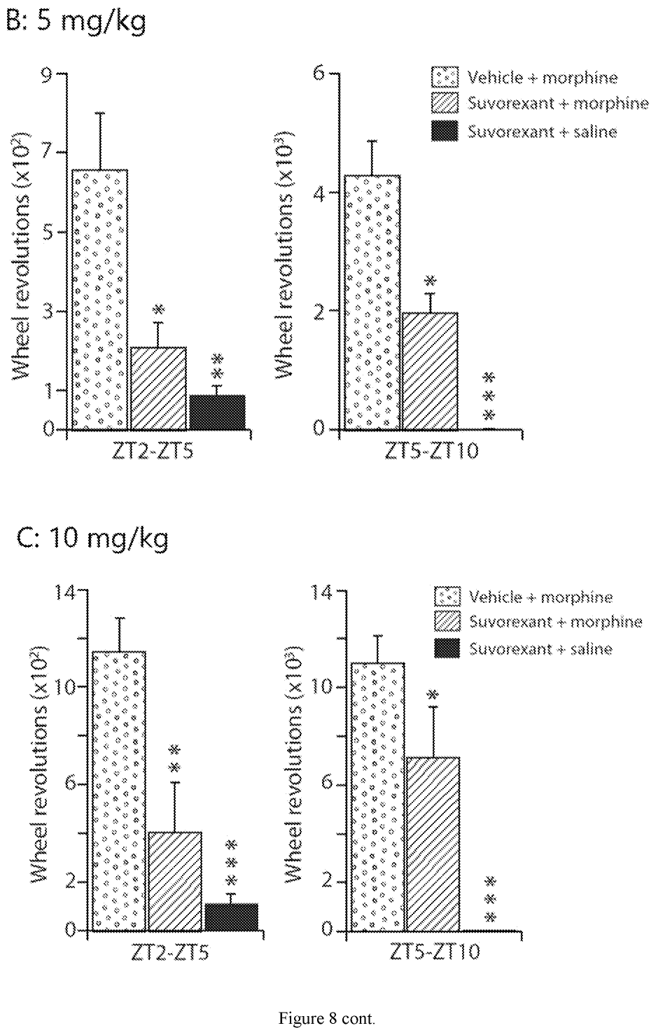

Morphine anticipation, which is an indicator of morphine addiction was evaluated when given alone or in combination with suvorexant. Three groups of 6 mice at 5 mg/kg of morphine and three groups of 6 mice at 10 mg/kg of morphine were studied. All groups were given an oral administration of vehicle or vehicle+suvorexant at ZT 4 (10 AM) (note increased running linked to the handling of the mice for gavage (oral) administration of suvorexant after this time point in morphine groups) followed 1 hour later by a SC injection of saline or morphine in saline. This was continued for 14 days. One group was given vehicle, followed 1 hour later by morphine, 5 mg/kg. A second group was given vehicle with 30 mg/kg suvorexant, followed 1 hour later with a 10 mg/kg morphine injection. The third group was given vehicle with 30 mg/kg suvorexant alone, followed 1 hour later by saline injection. FIG. 8A shows wheel running averaged over the last 12 days of the 14-day study periods for the three experimental groups after 5 mg/kg of morphine or suvorexant or both. Anticipatory running is seen in the vehicle+morphine group (dotted line) starting at ZT2 (8 AM, 2 hours after light on pulse). Running further increased after morphine injection in the vehicle +morphine group at ZTS. The anticipatory activity was completely absent in animals given suvorexant in vehicle followed by 5 mg/kg of morphine (dashed line). Suvorexant also greatly reduced running after morphine injection from ZT 5-8 (11 AM-2 PM) compared to vehicle+morphine alone, indicating, again, a major dampening of suvorexant on morphine induced motor excitation by blocking Hcrt receptors. There was no substantial “anticipatory” activity or any other activity from ZT0-12 when suvorexant in vehicle was given followed by saline injection (solid line). This study was conducted with both 5 and 10 mg/kg doses of morphine with a virtually identical pattern of activity shown in both experiments (the 5 mg dose is shown in the line graphs and both 5 and 10 mg doses are shown in the bar graphs (FIG. 8B & FIG. 8C) which indicate total activity during two ZT intervals, for each of the two morphine doses used. Comparisons to vehicle-morphine condition, *P=0.05; **0.01, ***P=0.001. These results indicate that opioid (e.g., morphine) anticipatory activity, as well as opioid induced activity, is facilitated by Hcrt receptor activation, which can be blocked with an inhibitor such as suvorexant.

### REFERENCES

The following references are herein incorporated by reference in their entirety with the exception that, should the scope and meaning of a term conflict with a definition explicitly set forth herein, the definition explicitly set forth herein controls:
1. T. C. Thannickal et al., Sci Transl Med 10, pii: eaao4953 (2018).
2. N. Assar, D. Mahmoudi, Z. Mousavi, S. Zarrabian, A. Haghparast, Behavioural Brain Research 373, 112090 (2019).
3. A. Mohammadkhani, M. H. James, C. B. Pantazis, G. Aston-Jones, Brain Research 146461 (2019).
4. M. Lupina et al., Molecular Neurobiology https://doi.org/10.1007/s12035-018-0993-0, (2018).
5. D. A. Perrey, Y. Zhang, Brain Res. 10 (2018).
6. P. Dowling, F. Klinker, F. Amaya, W. Paulus, D. Liebetanz, The Journal of Nutrition 139, 2087 (2009).
7. P. Dowling et al., The Journal of Neuroscience 31, 70 (2011).
8. N. D. Volkow, A. T. McLellan, New England Journal of Medicine 374, 1253 (2016).
9. S. Weiner, O. Baker, D. Bernson, J. Schuur, Annals of Emergency Medicine 70, 402 (2017).
10. P. S. Ostling et al., Curr Pain Headache Rep. 22, 32 (2018).
11. K. C. Chang et al., Int J Drug Policy. 43:1-6. doi: 10.1016/j.drugpo.2016.12.003. Epub; % 2017 Feb. 1, 1 (2017).
12. Editors, Lancet. 387, 404 (2016).
13. Y. Li, A. N. van den Pol, Journal of Neuroscience 28, 2814 (2008).
14. C.D.C., Centers for Disease Control and Prevention (2017).
15. C. Bass, G. Yates, Med Sci Law. 58, 147 (2018).
16. R. Berger, C. Pulido, J. Lacro, S. Groban, S. Robinson, J Addict. Med. 8, 415 (2014).
17. S. Conroy, D. Hill, BMJ Case. Rep. 2014. pii: bcr-2014-207458. doi: 10.1136/bcr-2014-207458, bcr (2014).
18. C. Dever, J Trauma Nurs. 24, 292 (2017).
19. M. Frei, Aust. Fam. Physician. 39, 548 (2010).
20. C. A. Huxtable, L. J. Roberts, A. A. Somogyi, P. E. Maclntyre, Anaesth. Intensive Care. 39, 804 (2011).
21. M. M. Kelly, E. Reilly, T. Quinones, N. Desai, R. Rosenheck, Drug Alcohol Depend. 183:111-117. doi: 10.1016/j.drugalcdep.2017.10.017. Epub; % 2017 Dec. 2, 111 (2018).
22. H. Palis et al., Subst. Use. Misuse. 51, 763 (2016).
23. R. Parthvi, A. Agrawal, S. Khanijo, A. Tsegaye, A. Talwar, Am J Ther. 26, e380 (2019).
24. N. Sanger et al., Pain Physician. 21, E623 (2018).
25. D. L. Waszak, A. M. Mitchell, D. Ren, L. A. Fennimore, J Emerg. Nurs. 44, 336 (2018).
26. K. T. White, T. R. Dillingham, M. Gonzalez-Fernandez, L. Rothfield, Am J Phys Med Rehabil. 88, 995 (2009).
27. F. Del Bello et al., ACS Med Chem Lett 4, 875 (2013).
28. P. E. Lutz et al., Neuropsychopharmacology 39, 2694 (2014).
29. J. Shi et al., Am J Drug Alcohol Abuse 35, 267 (2009).
30. Y. Zhu, C. F. Wienecke, G. Nachtrab, X. Chen, Nat. 530, 219 (2016).
31. M. J. Christie, Br J Pharmacol 2008 Apr. 14, 384 (2008).
32. S. C. Sigmon et al., Am J Drug Alcohol Abuse 38, 187 (2012).
33. A. McLellan, D. C. Lewis, C. P. O'Brien, H. D. Kleber, JAMA 284, 1689 (2000).
34. N. D. Volkow et al., Sci Transl Med 10, (2018).
35. R. McGregor, M. -F. Wu, G. Barber, L. Ramanathan, J. M. Siegel, Journal of Neuroscience 31, 15455 (2011).
36. M. F. Wu, R. Nienhuis, N. Maidment, H. A. Lam, J. M. Siegel, J Neurosci. 31, 6305 (2011).
37. M. F. Wu, R. Nienhuis, N. Maidment, H. A. Lam, J. M. Siegel, Arch. ital. Biol. 149, 492 (2011).
38. B. Y. Mileykovskiy, L. I. Kiyashchenko, J. M. Siegel, Neuron 46, 787 (2005).
39. L. I. Kiyashchenko et al., J Neurosci 22, 5282 (2002).
40. C. Baimel et al., Br J Pharmacol 172, 334 (2015).
41. E. J. Nestler et al., Neuron. 34, 13 (2002).
42. S. L. Borgland et al., Journal of Neuroscience 29, 11215 (2009).
43. D. Georgescu et al., J Neurosci 23, 3106 (2003).
44. M. H. James, S. V. Mahler, D. E. Moorman, G. Aston-Jones, Curr Top. Behav Neurosci. 33:247-281. doi: 10.1007/7854_2016_57, 247 (2017).
45. E. J. Nestler, Dialogues Clin Neurosci 15, 431 (2013).
46. G. Aston-Jones et al., Brain Research 1314, 74 (2010).
47. B. Boutrel, De Lecea L., Physiol Behay. 93, 947 (2008).
48. A. M. Blouin et al., Nature Communications 4:1547, 1547 (2013).
49. C. Peyron et al., Nat. Med. 6, 991 (2000).
50. T. C. Thannickal et al., Neuron. 27, 469 (2000).
51. A. Ponz et al., Ann Neurol. 67, 190 (2010).
52. M. J. Lee et al., Sleep Medicine 39, 95.
53. S. E. H. Nordstrand et al., Sleep zsz008 (2019).
54. M. Lutter et al., Journal of Neuroscience 28, 3071 (2008).
55. D. Beitner-Johnson, X. Guitart, E. J. Nestler, J Neurosci. 12, 2165 (1992).
56. D. Beitner-Johnson, X. Guitart, E. J. Nestler, J Neurochem. 61, 1766 (1993).
57. E. Mignot et al., Sleep Res 24, 298 (1995).
58. B. Schilstrom, H. M. Svensson, T. H. Svensson, G. G. Nomikos, Neuroscience 85, 1005 (1998).
59. F. Sarti, S. L. Borgland, V. N. Kharazia, A. Bonci, Eur J Neurosci. 26, 749 (2002).
60. S. Farahimanesh, S. Zarrabian, A. Haghparast, Neuropeptides. 10 (2017).
61. F. J. Meye, R. van Zessen, M. P. Smidt, R. A. H. Adan, G. M. J. Ramakers, Journal of Neuroscience 32, 16120 (2012).
62. G. B. Stefano, R. M. Kream, Int J Mol. Med. 20, 837 (2007).
63. C. Peyron et al., J Neurosci 18, 9996 (1998).
64. E. M. Anderson et al., Proc Natl Acad Sci USA. 114, 9469 (2017).
65. C. Y. Ho, K. C. Berridge, Neuropsychopharmacology. 10 (2013).
66. L. J. Sim-Selley et al., Neuropharmacology. 61, 1470 (2011).
67. M. Stoeber et al., Neuron 98, 963 (2018).
68. N. S. Cai et al., J Clin Invest. 129, 2730 (2019).
69. F. Torrealba, M. Yanagisawa, C. B. Saper, Neuroscience 119, 1033 (2003).
70. J. W. Muschamp et al., Proc Natl Acad Sci USA 111, E1648 (2014).
71. A. M. Blouin et al., Neurology 65, 1189 (2005).
72. A. Crocker et al., Neurology 65, 1184 (2005).
73. A. Flores, R. Maldonado, F. Berrendero, Biol Psychiatry. 75, 499 (2014).
74. H. Abdollahi, M. Ghaemi-Jandabi, H. Azizi, S. Semnanian, Brain Research 1646, 91 (2016).
75. M. Alcaraz-Iborra et al., Front Behav Neurosci. 11:186. doi: 10.3389/fnbeh.2017.00186. eCollection; % 2017, 186 (2017).
76. M. Fakhari, H. Azizi, S. Semnanian, Neuroscience 363, 1 (2017).
77. B. Hooshmand, H. Azizi, M. Javan, S. Semnanian, Neurosci. Lett. 636:276-281. doi: 10.1016/j.neulet.2016.10.051. Epub; % 2016 Nov. 2, 276 (2017).
78. D. E. Moorman, M. H. James, E. A. Kilroy, G. Aston-Jones, Brain Res. 1654, 34 (2017).
79. M. Kakizaki et al., iScience 2019 Sep. 9, 1 (2019).
80. K. Lei et al., Front Neurosci. 13:88. doi: 10.3389/fnins.2019.00088. eCollection; % 2019, 88 (2019).
81. S. Nishino et al., Ann. Neurol 50, 381 (2001).
82. M. A. Brown, C. Guilleminault, Curr Pharm. Des. 17, 1430 (2011).
83. C. Guilleminault, M. T. Cao, in Principles and Practice of Sleep Medicine, M. H. Kryger, T. Roth, W. C. Dement, Eds. (Elsevier Saunders, Missouri, 2011), pp. 957-968.
84. L. Barateau et al., Sleep. sp (2015).
85. Y. Dauvilliers, R. Lopez, M. Ohayon, S. Bayard, BMC Med 11, 78 (2013).
86. Y. Dauvilliers, J. M. Siegel, R. Lopez, Z. A. Torontali, J. H. Peever, Nat Rev Neurol 10, 386 (2014).
87. G. C. Harris, M. Wimmer, J. F. Randall-Thompson, G. Aston-Jones, Behav Brain Res. 183, 43 (2007).
88. S. Nishino, E. Mignot, Handb. Clin Neurol. 99:783-814, 783 (2011).
89. H. Jalal et al., Science 361, eaau1184 (2018).
90. C. Turner et al., Drug and Alcohol Dependence 185, 305 (2018).
91. S. Darke, S. Kaye, J. Duflou, J. Lappin, Suicide Life Threat Behav (2018).
92. S. Bayard, Y. A. Dauvilliers, Frontiers in Behavioral Neuroscience 7, (2013).
93. G. P. Galloway et al., Addiction. 92, 89 (1997).
94. A. Ponz et al., Ann Neurol. 67, 394 (2010).
95. S. Schwartz et al., Brain 131, 514 (2007).
96. K. A. Richardson, G. Aston-Jones, J Neurosci. 32, 3809 (2012).
97. G. C. Harris, M. Wimmer, G. Aston-Jones, Nat. 437, 556 (2005).
98. G. C. Harris, G. Aston-Jones, Trends Neurosci. 29, 571 (2006).
99. R. Sharf, M. Sarhan, R. J. DiLeone, Biol Psychiatry. 64, 175 (2008).
100. R. Sharf, D. J. Guarnieri, J. R. Taylor, R. J. DiLeone, Brain Res. 1317, 24 (2010).
101. N. Steiner et al., Neuropharmacology 133, 395 (2018).
102. R. M. Chemelli et al., Cell 98, 437 (1999).
103. T. E. Scammell, J. T. Willie, C. Guilleminault, J. M. Siegel, Sleep 32, 111 (2009).
104. F. Pizza et al., Sleep Med. 15, 262 (2014).
105. A. Alakuijala, T. Sarkanen, M. Partinen, Sleep Medicine 16, 39 (2015).
106. T. Verstraeten et al., Hum. Vaccin. Immunother. 1 (2015).
107. M. S. Schinkelshoek, G. J. Lammers, R. Fronczek, Sleep zsz009 (2019).
108. S. Tabuchi et al., J Neurosci 34, 6495 (2014).
109. J. John et al., Ann Neurol. 74, 786 (2013).
110. S. Chowdhury et al., bioRxiv 512947 (2019).
111. S. Tabuchi et al., J Neurosci 34, 6495 (2014).
112. W. J. Giardino et al., Nat Neurosci 2018 Jul. 23, 1084 (2018).
113. A. Inutsuka et al., Sci Rep 6, 29480 (2016).
114. H. Azizi, J. Mirnajafi-Zadeh, K. Rohampour, S. Semnanian, Neuroscience Letters 482, 255 (2010).
115. M. Ghaemi-Jandabi, H. Azizi, S. M. Ahmadi-Soleimani, S. Semnanian, Brain Res Bull. 130:107-111. doi: 10.1016/j.brainresbull.2017.01.010. Epub; % 2017 Jan. 16, 107 (2017).
116. M. J. Christie, J. T. Williams, P. B. Osborne, C. E. Bellchambers, Trends in Pharmacological Sciences 18, 134 (1997).
117. M. S. Mazei-Robison, E. J. Nestler, Cold Spring. Harb. Perspect. Med. 2, doi: 10.1101/cshperspect.a012070 (2012).
118. E. J. Nestler, Eur. J Pharmacol. 753:66-72. doi: 10.1016/j.ejphar.2014.10.034. Epub; % 2014 Nov. 7, 66 (2015).
119. R. McGregor, L. Shan, M. F. Wu, J. M. Siegel, PLoS ONE 12, (2017).
120. L. Sklair-Tavron et al., Proc Natl Acad Sci U.SA. 93, 11202 (1996).
121. J. R. Shoblock, N. T. Maidment, Neuropsychopharmacology. 31, 171 (2006).
122. P. D. Skoubis, H. A. Lam, J. Shoblock, S. Narayanan, N. T. Maidment, Eur J Neurosci. 21, 1379 (2005).
123. S. Narayanan et al., Neuroscience. 124, 241 (2004).
124. J. R. Shoblock, N. T. Maidment, Neuroscience. 149, 642 (2007).
125. A. S. James et al., Behavioural Brain Research 256, 279 (2013).
126. M. H. James et al., Biol Psychiatry doi: 10.1016/j.biopsych.2018.07.022. [Epub ahead of print] (2019).
127. C. V. Howard, M. G. Reed, Unbiased Stereology: Three dimensional measurement in microscopy (Springer-Verlag, New York, 1998).
128. T. C. Thannickal, J. M. Siegel, R. Y. Moore, Brain Pathol. 13, 340 (2003).
129. D. Gerashchenko et al., Exp. Neurol. 184, 1010 (2003).
130. L. I. Perrotti et al., Synapse. 62, 358 (2008).
131. J. A. McHenry et al., Behay. Neurosci. 130, 469 (2016).
132. C. Blanco-Centurion et al., IBRO Reports 4, 44 (2018).
133. H. Azhdari-Zarmehri, M. H. Esmaeili, M. Sofiabadi, H. Haghdoost-Yazdi, Pharmacology Biochemistry and Behavior 112, 64 (2013).
134. K. K. Szumlinski et al., Genes, Brain and Behavior n/a, e12609 (2019).
135. S. Y. -S. Khoo, R. M. Brown, CNS Drugs 28, 713 (2014).
136. C. J. Winrow et al., Neuropharmacology 62, 978 (2012).
137. R. J. Rodgers et al., Eur. J Neurosci. 13, 1444 (2001).
138. R. J. Rodgers, F. L. Wright, N. F. Snow, L. J. Taylor, Behavioural Brain Research 243, 213 (2013).
139. A. J. Lawrence, M. S. Cowen, H. J. Yang, F. Chen, B. Oldfield, Br J Pharmacol 2006 Jun. 5, 752 (2006).
140. S. Tabuchi et al., J Neurosci 34, 6495 (2014).
141. R. Maldonado et al., Science 273, 657 (1996).
142. Q. Zhang et al., Neurosci. Lett. 614:70-6. doi: 10.1016/j.neulet.2015.11.052. Epub; % 2015 Dec. 3, 70 (2016).
143. S. Tabuchi et al., J Neurosci 34, 6495 (2014).
144. V. Zachariou et al., Proc Natl Acad Sci USA 100, 9028 (2003).
145. J. A. Gonzalez, J. H. M. Prehn, Neuroscience 369, 183 (2018).
146. S. Li, P. Franken, A. Vassalli, Sci Rep 8, 15474 (2018).
147. M. Hooshmandi et al., Pharmacology Biochemistry and Behavior 158, 39 (2017).
148. S. M. Ahmadi-Soleimani, M. Ghaemi-Jandabi, H. Azizi, S. Semnanian, Neuroscience Letters 558, 62 (2014).
149. R. J. Smith, G. Aston-Jones, European Journal of Neuroscience 35, 798 (2012).
150. N. N. Chaijale et al., Neuropsychopharmacology. 38, 1833 (2013).
151. K. M. Jaremko et al., Prog Neuropsychopharmacol. Biol Psychiatry. 50:53-65. Epub@2013 Dec. 12 doi:10.1016/j.pnpbp.2013.12.003, 53 (2014).
152. A. Kaeidi et al., PLoS ONE. 10, doi (2015).
153. R. J. Valentino, B. E. Van, Eur J Pharmacol. 583, 194 (2008).
154. J. M. Delfs, Y. Zhu, J. P. Druhan, G. Aston-Jones, Nat. 403, 430 (2000).
155. C. Blanco-Centurion, D. Gerashchenko, P. J. Shiromani, J Neurosci. 27, 14041 (2007).
156. W. B. Solecki et al., Behavioural Brain Research 360, 146 (2019).
157. L. Zhang et al., EMBO Mol Med 2017 Aug. 18, 1521 (2017).
158. J. R. Parkitna et al., International Journal of Neuropsychopharmacology 15, 1457 (2012).
159. A. J. Lopez et al., Addict Biol. 10 (2018).
160. S. Kang, J. Li, A. Bekker, J. H. Ye, Neuropharmacology. 129:47-56. doi: 10.1016/j.neuropharm.2017.11.013. Epub@2017 Nov. 8, 47 (2018).
161. K. Curtis et al., Am J Addict. 26, 751 (2017).
162. F. J. Meye, M. Trusel, M. Soiza-Reilly, M. Mameli, Pharmacol Biochem Behay. 162:87-93. doi: 10.1016/j.pbb.2017.08.007. Epub@2017 Aug. 24, 87 (2017).
163. I. McLaughlin, J. A. Dani, M. DeBiasi, J Neurochem. 142 Suppl 2:130-143. doi: 10.1111/jnc.14008, 130 (2017).
164. J. Wang et al., Neurosci Lett. 653:64-70. doi: 10.1016/j.neulet.2017.05.027. Epub@2017 May 17, 64 (2017).
165. J. Li et al., Alcohol Clin Exp Res. 41, 637 (2017).
166. K. M. Velasquez, D. L. Molfese, R. Salas, Front Hum. Neurosci. 8:174. doi: 10.3389/fnhum.2014.00174. eCollection@2014, 174 (2014).
167. D. Ferguson et al., J Neurosci. 33, 16088 (2013).
168. C. Perez-Leighton, M. R. Little, M. Grace, C. Billington, C. M. Kotz, Am J Physiol Regul Integr Comp Physiol. 312, R338 (2017).
169. M. Sustkova-Fiserova et al., Int J Mol Sci. 18, doi (2017).
170. S. Ferenczi et al., Neurochem Int 57, 323 (2010).
171. J. Kaufling et al., Br J Pharmacol. 161, 1677 (2010).
172. H. E. Nye, E. J. Nestler, Mol Pharmacol. 49, 636 (1996).
173. J. C. Lin et al., Pain Med 2015 Dec. 22, 1497 (2016).
174. C. Baimel, S. L. Borgland, The Journal of Neuroscience 35, 7295 (2015).
175. M. Chen et al., eLife. 4:. doi:10.7554/eLife.09275, doi (2015).
176. E. A. Heller et al., J Neurochem 132, 243 (2015).
177. W. Shi et al., NeuroToxicology 51, 51 (2015).
178. L. Zarepour, Z. Fatahi, A. Sarihi, A. Haghparast, Neuropeptides. 48, 179 (2014).
179. R. Z. Goldstein, N. D. Volkow, Nature Reviews Neuroscience 12, 652 (2011).
180. C. Liu, X. Fang, Q. Wu, G. Jin, X. Zhen, Neuropharmacology 95, 299 (2015).
181. B. A. Baldo, Trends Neurosci 39, 366 (2016).
182. F. Wang et al., Physiol Res. 65, 701 (2016).
183. R. J. Smith, G. Aston-Jones, Brain Struct Funct 2008 Jul. 24, 43 (2008).
184. R. J. Valentino, N. D. Volkow, Neuropsychopharmacology (2019).
185. J. M. Siegel, P. R. Manger, R. Nienhuis, H. M. Fahringer, J. D. Pettigrew, Sleep Research 26, 177 (1997).
186. A. N. van den Pol, X. B. Gao, K. Obrietan, T. S. Kilduff, A. B. Belousov, J. Neurosci 18, 7962 (1998).
187. V. Casado-Anguera et al., Biochem. Soc. Trans. 44, 595 (2016).
188. E. Rossoni et al., PLoS Comput Biol 4, e1000123 (2008).
189. J. Qiu et al., eLife 5, e16246 (2016).
190. W. D. Branton, S. Arch, T. Smock, E. Mayeri, 75, 5732 (1978).
191. J. John et al., J Physiol 560, 587 (2004).
192. M. F. Wu, J. John, N. Maidment, H. A. Lam, J. M. Siegel, Am J Physiol Regul Integr Comp Physiol 283, R1079 (2002).

All scientific and technical terms used in this application have meanings commonly used in the art unless otherwise specified.

Abbreviations: DOX=doxycycline; DTA-Hcrt=mice in which Hcrt neurons can be killed by removing DOX; DTA-Hcrt-WT=DTA-Hcrt controls—DOX never removed; DTA-Hcrt-depleted=the desired percent of Hcrt neurons is killed by varying the period of DOX removal; F=female; Hcrt=hypocretin=orexin; Hcrt-KO=Hcrt not synthesized but dynorphin, glutamate, and Narp remain in “former Hcrt” neurons; LC=locus coeruleus; M=male; OUD=opioid use disorder; SQ=subcutaneous; TH=tyrosine hydroxylase; VTA=ventral tegmental area; WT=wild type; hypocretin (Hcrt) neurons=Hcrt cells=neurons that produce hypocretin; ZT=Zeitgeber Time, hours after lights on (e.g., ZT1=1 hour after lights on).

As used herein, a “pain” refers to acute pain and chronic pain.

As used herein, an “opioid” refers to a compound that acts on opioid receptors to result in an analgesic effect. Exemplary opioids include: opioid peptides such as endorphins, enkephalins, dynorphins, and endomorphins; opium alkaloids such as codeine, morphine, thebaine, oripavine, and papaveretum; esters of morphine such as diacetylmorphine (morphine diacetate; heroin), nicomorphine (morphine dinicotinate), dipropanoylmorphine (morphine dipropionate), diacetyldihydromorphine, acetylpropionylmorphine, desomorphine, methyldesorphine, and dibenzoylmorphine; ethers of morphine such as dihydrocodeine, ethylmorphine, and heterocodeine; synthetic alkaloids such as buprenorphine, etorphine, hydrocodone, hydromorphone, oxycodone, and oxymorphone; and synthetic opioids such as anilidopiperidines (e.g., fentanyl, alphamethylfentanyl, alfentanil, sufentanil, remifentanil, carfentanyl, ohmefentanyl), phenylpiperidines (e.g., pethidine (meperidine), ketobemidone, MPPP, allylprodine, prodine, PEPAP, promedol), diphenylpropylamine derivatives (e.g., propoxyphene, dextropropoxyphene, dextromoramide, bezitramide, piritramide, methadone, dipipanone, levomethadyl acetate, difenoxin, diphenoxylate, loperamide), benzomorphan derivatives (e.g., dezocine, pentazocine, phenazocine), oripavine derivatives (e.g., buprenorphine, dihydroetorphine, etorphine), morphinan derivatives (e.g., butorphanol, nalbuphine, levorphanol, levomethorphan, racemethorphan), lefetamine, menthol, meptazinol, mitragynine, tilidine, tramadol, tapentadol, eluxadoline, AP-237, 7-hydroxymitragynine; and the like. In some embodiments, the opioid is morphine.

As used herein, a “hypocretin/orexin receptor antagonist” refers to a compound that inhibits or reduces the signaling of hypocretin/orexin receptors, e.g., a hypocretin 1 receptor and/or a hypocretin 2 receptor, by inhibiting agonists (e.g., hypocretin/orexin) from binding thereto. Hypocretin/orexin receptor antagonists include suvorexant, almorexant, EMPA, filorexant, JNJ-10397049, lemborexant, MIN-202, MK-1064, MK-8133, nemorexant, RTIOX-276, SB-334867, SB-408124, SB-649868 (CAS No. 380899-24-1), TCS-OX2-29, (3,4-dimethoxyphenoxy) alkylamino acetamides, Compound 1 m (Fujimoto T, et al. (2011) Bioorganic & Medicinal Chemistry Letters. 21 (21): 6414-6), and the like. In some embodiments, the hypocretin/orexin receptor antagonist is suvorexant.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “sample” is used in its broadest sense and includes specimens and cultures obtained from any source, as well as biological samples and environmental samples. Biological samples may be obtained from animals (including humans) and encompass fluids, solids, tissues, and gases. Biological samples include blood products, such as plasma, serum, and the like. A biological sample can be obtained from a subject using methods in the art.

As used herein, an “effective amount” refers to a dosage or amount sufficient to produce a desired result. The desired result may comprise an objective or subjective change as compared to a control in, for example, in vitro assays, and other laboratory experiments.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A composition comprising one or more opioids and one or more hypocretin/orexin receptor antagonists.

2. The composition according to claim 1, wherein the one or more opioids is provided in a therapeutically effective amount for treating, inhibiting, or reducing pain in a subject and/or the one or more hypocretin/orexin receptor antagonists is provided in a therapeutically effective amount for inhibiting or reducing the likelihood that the subject will develop an addiction to the one or more opioids.

3. The composition according to claim 1, wherein the one or more opioids is morphine and/or the one or more hypocretin/orexin receptor antagonists is suvorexant.

4. The composition according to claim 1, wherein the composition comprises about 1-10 mg of the one or more opioids.

5. The composition according to claim 1, wherein the composition comprises about 1-10 mg of the one or more hypocretin/orexin receptor antagonists.

6. The composition according to claim 1, wherein the composition is an oral formulation or an intravenous formulation.

7. The composition according to claim 1, further comprising a pharmaceutically acceptable vehicle.

8. A method for treating, inhibiting, or reducing pain in a subject, which comprises administering to the subject one or more opioids in combination with one or more hypocretin/orexin receptor antagonists.

9. The method of claim 8, wherein the one or more hypocretin/orexin receptor antagonists is administered before, during, or after the administration of one or more opioids.

10. The method according to claim 8, wherein the one or more opioids is morphine and/or the one or more hypocretin/orexin receptor antagonists is suvorexant.

11. The method according to claim 8, wherein about 0.05-0.15 mg of the one or more opioids per kg weight of the subject is administered.

12. The method according to claim 8, wherein about 0.05-0.15 mg of the one or more hypocretin/orexin receptor antagonists per kg weight of the subject is administered.

13. The method according to claim 8, wherein the one or more opioids and the one or more hypocretin/orexin receptor antagonists is administered to the subject in the form of a composition comprising one or more opioids and one or more hypocretin/orexin receptor antagonists.

14. A method of inhibiting or reducing an increase in the amount of hypocretin and/or an increase in the amount of hypocretin neurons, which increases are caused by administration of one or more opioids, in a subject, which comprises administering to the subject one or more hypocretin/orexin receptor antagonists before, during, or after the administration of the one or more opioids.

15. A kit comprising one or more opioids packaged together with one or more hypocretin/orexin receptor antagonists.
